(19) United States
(12) Patent Application Publication
(10) Pub. No.: US 2023/0172874 A1
(43) Pub. Date: Jun. 8, 2023

(54) USE OF CANNABIDIOL FOR THE TREATMENT OF INSOMNIA ASSOCIATED TO PAIN

(71) Applicants: Gabriella GOBBI, Montreal (CA); Danilo DE GREGORIO, Milan (IT); Martha Graciela LÓPEZ-CANUL, Montreal (CA)

(72) Inventors: Gabriella GOBBI, Montreal (CA); Danilo DE GREGORIO, Milan (IT); Martha Graciela LÓPEZ-CANUL, Montreal (CA)

(21) Appl. No.: 18/061,810

(22) Filed: Dec. 5, 2022

Related U.S. Application Data
(60) Provisional application No. 63/264,970, filed on Dec. 6, 2021.

(30) Foreign Application Priority Data
Apr. 19, 2022 (CA) 3156260

(51) Int. Cl. A61K 31/05; A61K 31/454; A61P 25/20; A61P 21/00

(52) U.S. Cl. CPC A61K 31/05; A61K 31/454; A61P 25/20; A61P 21/00

(57) ABSTRACT

Chronic pain or neuropathic pain (NP) is associated with sleep disorders, and in turn sleep disorders increase pain such as neuropathic pain. A limited number of drugs are available for treating NP associated insomnia, and side effects are common. The present application describes methods and uses based on CBD and analogs/derivatives thereof for the management of insomnia and/or sleep disorders associated to all types of chronic and/or NP. The present application also relates to the management of disorders associated with REM sleep using a CB₁ inhibitor. The present application also relates to the use of sleep electroencephalogram (EEG) analysis for the assessment of chronic pain/NP.

### CROSS-REFERENCE TO RELATED APPLICATIONS

This application claims the benefit of Canadian Patent Application No. 3,156,260, filed on Apr. 19, 2022, and of U.S. Provisional Patent Application No. 63/264,970, filed on Dec. 6, 2021, which are incorporated herein by reference.

### FIELD OF THE INVENTION

The present disclosure relates to the management of pain and/or sleep disorders, and more particularly to the treatment of insomnia associated to neuropathic and/or chronic pain, to the diagnosis of neuropathic pain, and to the treatment of disorders associated with abnormal rapid eye movement (REM) sleep such as narcolepsy.

### BACKGROUND OF THE INVENTION

Neuropathic pain (NP) is a major health problem that results in a high degree of suffering, physical and psychosocial impairments and exorbitant health care costs. NP is a chronic pain disorder resulting from damage to the nervous system, which can be caused by medical conditions such as cancer, diabetes, infection, or traumatic injury¹⁻⁴. NP represents a major economic burden and considerably impairs patients' quality of life^{5,6}. Individuals with NP display distinct sensory symptoms that can coexist in multiple combinations^{2,6,7}. Between 15 and 50% of NP patients are affected by allodynia (pain caused by normally innocuous stimuli) and hyperalgesia (extreme pain response to a stimulus that normally causes pain)². These cardinal and intractable symptoms result from peripheral sensitization and maladaptive central change^{2,8}. Despite tremendous progress, our understanding of the pathological mechanisms underlying allodynia and hyperalgesia is still incomplete, and current treatments are largely ineffective^{1,8,9}. Psychiatric and medical comorbidities usually co-occur, in fact, patients who suffer from chronic pain often experience depression and insomnia^{4,10,11}.

Sleep is a regulated biological state characterized by a reduction in voluntary motor activities, attenuated response to stimulation, and stereotypic posture. It is conserved across species and essential for survival¹². The mammalian sleep-wake cycle progresses in three stages distinguished by electroencephalographic (EEG) activity and muscular movements measured by electromyography (EMG). Wakefulness stage is characterized by Alpha and Beta waves, (8-13 and 13-30 Hz. respectively) and sustained EMG signals; following the transition into Non-Rapid Eye Movement (NREM) sleep, the EEG signal increases in voltage and decreases in frequency into Delta waves (0.5-4 Hz), and the muscular movement is also decreased; lastly, the REM sleep is characterized by fast, low amplitude EEG oscillation theta waves (t6.0-9.0 Hz) and muscle atonia¹³. Multiple neurotransmitters are involved in the modulation of the sleep-wake cycle.

Between 67% and 88% of people with chronic pain report insomnia as a major source of distress^{11,14,15.} Insomnia is the most commonly noted sleep disorder associated with chronic pain. A large meta-analysis noted a high prevalence of other sleep disruptions as well, with the 3 most common being insomnia at 72%, restless leg syndrome with a prevalence of 32%, and obstructive sleep apnea with a prevalence of 32% (Mathias J L, Cant M L, Burke A L J. Sleep disturbances and sleep disorders in adults living with chronic pain: a meta-analysis. Sleep Med. 2018; 52:198-210). The three most commonly observed sleep disorders added up to greater than 100%, highlighting the fact that chronic pain can be associated with more than 1 type of sleep disturbance at a time. Importantly, pain-associated sleep disturbance also can affect patients' response to pain. As sleep stages are compromised, daytime pain increases through a process of hyperalgesia, in which lower pain stimuli result in higher pain perception and greater pain-associated impairment than would be expected otherwise.

Pharmacological strategies directed at nociceptive mechanisms do not yield improvements in sleep in people with chronic pain⁴¹. A few numbers of drugs are available for treating NP associated insomnia, and side effects are common. Moreover, some have serious side effects including risk of addiction and sleep-related breathing difficulties and apnea leading to increased risk of deaths^{16,17}.

Studies on the effect of cannabidiol (CBD) on sleep have led to inconsistent results. A preliminary study revealed that acute systemic administration of 20 mg/kg of CBD to naïve animals reduced slow-wave sleep (SWS) latency with no significant effect on sleep duration and sleep parameters, whereas single doses of 40 mg/kg not only decreased SWS latency but also increased SWS without affecting REM sleep¹⁸. In a preclinical study, Chagas and colleagues confirmed this trend. CBD doses of 10 and 40 mg/kg significantly increased the total percentage of sleep, however, the increase in SWS duration observed with CBD dose of 40 mg/kg was not statistically significant¹⁹. Conversely, Murillo-Rodriguez and colleagues provided experimental evidence about the wake-inducing proprieties of CBD. Intracerebroventricular injection of CBD (10 μg/5 μL) in healthy rats induced an increase in wakefulness and a decrease in REM sleep²⁰. Until now, no studies were carried out using CBD in models of neuropathic pain and co-morbid insomnia.

Narcolepsy is a chronic sleep disorder characterized by overwhelming daytime drowsiness and sudden attacks of sleep. Narcolepsy is typically associated with abnormalities in REM sleep, which may occur at any time of the day and very rapidly (within 15 minutes) after falling asleep, in people with narcolepsy. Current treatments for narcolepsy include stimulants, serotonin reuptake inhibitors (SSRIs) and serotonin and norepinephrine reuptake inhibitors (SNRIs), tricyclic antidepressants and sodium oxybate, but these treatments may be associated with various undesirable side effects.

Little is known about the role of CB1 antagonism on sleep. Santucci and colleagues²¹ demonstrated that the administration of the CB1 selective antagonist SR 141716A (rimonabant) dose-dependently increased the time spent in wakefulness, while reducing the time spent in NREM sleep and REM sleep.

There is thus a need for the development of novel approaches for the management of pain-associated insomnia and other sleep-related disorders such as narcolepsy.

### SUMMARY OF THE INVENTION

The present disclosure provides the following items 1 to 49:

1. A method for treating or managing insomnia or a sleep disorder associated to pain in a human subject in need thereof (e.g., in a subject suffering from or susceptible to the chronic pain condition and comorbid insomnia), the method comprising administering to the subject an effective amount of cannabidiol (CBD), a CBD analog, or a pharmaceutically acceptable salt or solvate thereof.
2. The method of item 1, wherein the pain is neuropathic pain.
3. The method of item 1 or 2, wherein the pain is post-herpetic (or post-shingles) neuralgia, reflex sympathetic dystrophy/causalgia (nerve trauma), components of cancer pain, phantom limb pain, entrapment neuropathy (e.g., carpal tunnel syndrome), peripheral neuropathy (widespread nerve damage), diabetic neuropathy, lower back pain, pain induced by chemotherapy (cisplatin, paclitaxel, vincristine, etc.) or radiotherapy, pain caused by HIV infection or AIDS, pain caused by central nervous system disorders (stroke, Parkinson's disease, multiple sclerosis, etc.), complex regional pain syndrome, nerve compression or infiltration by tumors.
4. The method of any one of items 1 to 3, wherein the CBD analog is cannabidiolic acid (CBDA), cannabidiol-3-monomethyl ether (CBDM-C₅), cannabidibutol (CBD-C₄), cannabidivarinic acid (CBDVA), cannabidivarin (CBDV), or cannabidiorcol (CBD-C₁).
5. The method of any one of items 1 to 4, wherein the method comprises administering to the subject an effective amount of CBD or a pharmaceutically acceptable salt or solvate thereof.
6. The method of item 5, wherein the method comprises administering to the subject an effective amount of CBD.
7. The method of any one of items 1 to 6, wherein the CBD, CBD analog, or pharmaceutically acceptable salt or solvate thereof is administered to the human subject at a dose corresponding to a dose of about 5 to about 20 milligrams/kg in rats.
8. The method of any one of items 1 to 7, wherein the total daily dose of the CBD, CBD analog, or pharmaceutically acceptable salt or solvate thereof administered to the human subject corresponding to a dose of about 5 to about 20 milligrams/kg in rats.
9. The method of any one of items 1 to 8, wherein the CBD, CBD analog, or pharmaceutically acceptable salt or solvate thereof is administered once-a-day or twice-a-day.
10. The method of any one of items 1 to 9, wherein the CBD, CBD analog, or pharmaceutically acceptable salt or solvate thereof is administered for at least a week.
11. The method of any one of items 1 to 10, wherein the CBD, CBD analog, or pharmaceutically acceptable salt or solvate thereof is administered as an immediate release formulation.
12. The method of any one of items 1 to 11, wherein the CBD, CBD analog, or pharmaceutically acceptable salt or solvate thereof is administered as a controlled release or extended release formulation.
13. The method of any one of items 1 to 12, wherein the CBD, CBD analog, or pharmaceutically acceptable salt or solvate thereof is administered parentally, for example intravenously (IV).
14. The method of any one of items 1 to 12, wherein the CBD, CBD analog, or pharmaceutically acceptable salt or solvate thereof is administered orally.
15. The method of item 14, wherein the CBD, CBD analog, or pharmaceutically acceptable salt or solvate thereof is formulated as an oral formulation.
16. The method of item 15, wherein the oral formulation is in a solid form or a liquid form.
17. The method of any one of items 1 to 16, wherein the method (a) increases REM sleep time, (b) increases non-REM (NREM) sleep and/or SWS time, and/or (c) decrease wakefulness time in the subject.
18. The method of item 17, wherein the method (a) increases rapid eye movement (REM) sleep time, (b) increases NREM sleep and/or SWS time, and (c) decreases wakefulness time in the subject.
19. A composition comprising cannabidiol (CBD), a CBD analog, or a pharmaceutically acceptable salt or solvate thereof for use in treating or managing insomnia or a sleep disorder associated to pain in a subject (e.g., in a subject suffering from or susceptible to the chronic pain condition and comorbid insomnia).
20. The composition for use according to item 19, wherein the pain is neuropathic pain.
21. The composition for use according to item 19 or 20, wherein the pain is post-herpetic (or post-shingles) neuralgia, reflex sympathetic dystrophy/causalgia (nerve trauma), components of cancer pain, phantom limb pain, entrapment neuropathy (e.g., carpal tunnel syndrome), peripheral neuropathy (widespread nerve damage), diabetic neuropathy, lower back pain, pain induced by chemotherapy (cisplatin, paclitaxel, vincristine, etc.) or radiotherapy, pain caused by HIV infection or AIDS, pain caused by central nervous system disorders (stroke, Parkinson's disease, multiple sclerosis, etc.), complex regional pain syndrome, nerve compression or infiltration by tumors.
22. The composition for use according to any one of items 19 to 21, wherein the CBD analog is cannabidiolic acid (CBDA), cannabidiol-3-monomethyl ether (CBDM-C₅), cannabidibutol (CBD-C₄), cannabidivarinic acid (CBDVA), cannabidivarin (CBDV), or cannabidiorcol (CBD-C₁).
23. The composition for use according to any one of items 19 to 22, wherein the composition comprises CBD or a pharmaceutically acceptable salt or solvate thereof.
24. The composition for use according to item 23, wherein the composition comprises CBD.
25. The composition for use according to any one of items 19 to 24, wherein the composition is for administration of the CBD, CBD analog, or pharmaceutically acceptable salt or solvate thereof at a dose from about 5 to about 20 milligrams/kg.
26. The composition for use according to any one of items 19 to 25, wherein the composition is for administration of the CBD, CBD analog, or pharmaceutically acceptable salt or solvate thereof at a total daily dose from about 5 to about 20 milligrams/kg.
27. The composition for use according to any one of items 19 to 26, wherein the composition is for administration of the CBD, CBD analog, or pharmaceutically acceptable salt or solvate thereof once-a-day or twice-a-day.
28. The composition for use according to any one of items 19 to 27, wherein the composition is for administration for at least a week.
29. The composition for use according to any one of items 19 to 28, wherein the composition is an immediate release formulation.
30. The composition for use according to any one of items 19 to 28, wherein the composition is a controlled release or extended release formulation.
31. The composition for use according to any one of items 19 to 30, wherein the composition is a parental, e.g., intravenous (IV), formulation.
32. The composition for use according to any one of items 19 to 30, wherein the composition is an oral formulation.
33. The composition for use according to item 32, wherein the oral formulation is in a solid form or a liquid form.
34. The composition for use according to any one of items 19 to 33, wherein the composition (a) increases REM sleep time, (b) increases non-REM (NREM) sleep and/or SWS time, and/or (c) decrease wakefulness time in the subject.
35. The composition for use according to item 34, wherein the composition (a) increases rapid eye movement (REM) sleep time, (b) increases NREM sleep and/or SWS time, and (c) decreases wakefulness time in the subject.
36. A method for identifying a subject suffering from insomnia or a sleep disorder associated to pain, the method comprising:
measuring at least one of the following parameters in the subject: rapid eye movement (REM) sleep time, non-REM sleep or SWS time, and wakefulness time; and
comparing the measured REM sleep time, non-REM sleep or SWS time, and/or wakefulness time to corresponding control or reference times;
   wherein a reduced (shorter) REM sleep time, reduced (shorter) non-REM sleep and/or SWS time and/or increased (longer) wakefulness time relative to the corresponding control or reference times is indicative that the subject suffers from insomnia or a sleep disorder associated to pain.
   37. The method of item 36, further comprising administering a suitable therapy against insomnia or the sleep disorder to the subject identified by the method of item 36.
   38. The method of item 37, wherein the therapy comprises the method defined in any one of items 1 to 18.
   39. Use of a cannabinoid receptor 1 (CB₁) inhibitor for the management of a disorder associated with abnormal rapid eye movement (REM) sleep in a subject.
   40. Use of a cannabinoid receptor 1 (CB₁) inhibitor for the manufacture of a medicament for the management of a disorder associated with abnormal rapid eye movement (REM) sleep in a subject.
   41. The use of item 39 or 40, wherein the abnormal REM sleep is excessive REM sleep.
   42. The use of any one of items 39 to 40, wherein the disorder is narcolepsy.
   43. The use of item 42, wherein the narcolepsy is type 1 narcolepsy.
   44. The use of item 42, wherein the narcolepsy is type 2 narcolepsy.
   45. The use of any one of items 39 to 41, wherein the disorder is a REM sleep behavior disorder (RBD).
   46. The use of item 45, wherein the RBD is associated with Lewy body dementia, Parkinson's disease or multiple system atrophy.
   47. The use of any one of items 39 to 46, wherein the CB₁ inhibitor is a CB₁ inverse agonist.
   48. The use of any one of items 39 to 47, wherein the CB₁ inhibitor is Rimonabant or an analog thereof.
   49. The use of item 48, wherein the CB₁ inhibitor is Rimonabant.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

FIGS. 1A-1C depict the chemical structure of CBD and of representative CBD analogs/derivatives.

FIGS. 2A and 2B show that CBD reduces mechanical allodynia in a dose-dependent manner. FIG. 2A: Time course of paw withdrawal threshold after von Frey filament stimulation in rats with SNI before (time 0) and after (0.5-7.5 hours) increasing doses of CBD (5, 10, and 20 mg/kg, intraperitoneally) in comparison with vehicle treated rats. The dashed line through the graph represents the threshold cut off (4 g) for allodynia in NP rats, in which values above the line are considered anti-allodynic. Data are expressed as mean±SEM, *p<0.05, **p<0.01, and ***p<0.001 vs. vehicle, by Bonferroni post hoc test. FIG. 2B: Area under the curve (AUC) of the antiallodynic effect of increasing doses of CBD. Data are expressed as mean±SEM, *p<0.05, **p<0.01, and ***p<0.001 vs. vehicle, by Bonferroni post hoc test.

FIGS. 3A-3C show that animals with neuropathic pain develop sleep perturbations. Animals with a NP condition develop sleep perturbations characterized by a decrease in REM (FIG. 3A), NREM (FIG. 3B) sleep and an increase in wakefulness (FIG. 3C). Data are expressed as mean±SEM, *p<0.05, **p<0.01, and ***p<0.001 vs. Naïve.

FIGS. 4A-4D show that CBD (20 mg/kg) restored the normal sleep-wake cycle in NP rats. FIG. 4A: CBD restored REM sleep in Neuropathic animals. Bonferroni post hoc comparison shows no difference between Naïve animals treated with vehicle and Neuropathic rats treated with CBD (20 mg/kg). Data are expressed as mean±SEM. *P<0.05, **P<0.01, and ***P<0.001 vs. NP+VEH. FIG. 4B: CBD restored NREM sleep in Neuropathic animals. Bonferroni post hoc comparison shows no difference between Naïve animals treated with vehicle and Neuropathic rats treated with CBD (20 mg/kg). Data are expressed as mean±SEM, (n=9). *P<0.05, **P<0.01, and ***P<0.001 vs. NP+VEH. FIG. 4C: CBD decreased wakefulness in neuropathic animals. In fact, Bonferroni post hoc comparison reveals no difference between Naïve animals treated with vehicle and neuropathic rats treated with CBD (20 mg/kg). Data are expressed as mean±SEM. *P<0.05, **P<0.01, and ***P<0.001 vs. NP+VEH. FIG. 4D: CBD attenuates the sleep fragmentation (repetitive short interruption of sleep) induced by neuropathy. Bonferroni post hoc comparison shows no difference between Sham operated animals treated with vehicle and Neuropathic rats treated with CBD (20 mg/kg). Data are expressed as mean±SEM. *P<0.05 vs. NP+VEH.

FIGS. 5A-5C show that WAY 100635 blocks the hypnotic effects of CBD. In neuropathic rats, the hypnotic effects of CBD (20 mg/kg) were fully prevented by the administration of the 5-HT1A receptor selective antagonist WAY (2 mg/kg). Moreover, WAY impaired NREM sleep (FIG. 5B) and increased wakefulness (FIG. 5C) in neuropathic rats relative to NP rats treated with vehicle. Data are expressed as mean±SEM, *p<0.05, **p<0.01, and ***p<0.001 vs. VEH, and ###P<0.001 vs. 20 mg/kg CBD by Bonferroni post hoc test.

FIGS. 6A-6C show the effects of Neuropathy+CBD (neuropathic animal plus CBD 20 mg/kg) vs. non-neuropathic rats+vehicle (Naïve+VEH), vs. Neuropathic animals+vehicle (Neuropathy+VEH) on the EEG power of delta, theta and sigma bands during NREM sleep. Data are expressed as mean (percentage of variation vs. vehicle)±S.E.M. *p<0.05 vs. vehicle.

FIGS. 7A-7C show the effects of Neuropathy+CBD (neuropathic animal plus CBD 20 mg/kg) vs. non-neuropathic rats+vehicle (Naïve+VEH), vs. Neuropathic animals+vehicle (Neuropathy+VEH) on the EEG power of delta, theta and sigma bands during REM sleep. Data are expressed as mean (percentage of variation vs. vehicle)±S.E.M. *p<0.05 vs. vehicle.

FIGS. 8A-8C show the effects of Neuropathy in animals treated with CBD, WAY 100635+CBD (WAY+CBD), WAY 100635 (WAY) alone and Vehicle (VEH) on the EEG power of delta, theta and sigma bands during NREM sleep. Data are expressed as mean (percentage of variation vs. vehicle)±S.E.M. *p<0.05 vs. vehicle.

#p<0.05 vs WAY+CBD.

FIGS. 9A-9C show the effects of Neuropathy in animals treated with CBD, WAY 100635+CBD (WAY+CBD), WAY 100635 (WAY) alone and Vehicle (VEH) on the EEG power of delta, theta and sigma bands during REM sleep. Data are expressed as mean (percentage of variation vs. vehicle)±S.E.M. *p<0.05 vs. vehicle.

FIGS. 10A-10C show the effect of the CB1 antagonist rimonabant (1 mg/kg) on NREM (FIG. 10A), REM (FIG. 10B) and wakefulness (FIG. 10C) in neuropathic animals. Data are expressed as mean±SEM, *p<0.05 vs. Naïve+Vehicle: **p<0.01 vs. Naïve+Vehicle, and ***p<0.001 vs. Naïve+vehicle; #p<0.05 vs. Neuropathy+vehicle

### DETAILED DESCRIPTION

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in various sources²²⁻²⁸.

In the studies described herein, the present inventors have demonstrated that animals with a NP condition develop sleep architecture perturbations characterized by a decrease in time of the non-rapid eye movement (NREM) sleep and rapid eye movement (REM) sleep, and an increase in time of wakefulness during the inactive phase of the day (night for humans and light for nocturnal rodents). It is shown that the systemic administration of CBD (5-20 mg/kg) reduces neuropathic pain (spared nerve injury-induced mechanical allodynia) and restores the impaired sleep in a dose-dependent manner in NP laboratory animals. The hypnotic effects of CBD were prevented by the 5-HT1A selective antagonist WAY 100635 (2 mg/kg). The present inventors have also demonstrated that treatment with the CB1 antagonist rimonabant decreases REM sleep in neuropathic animals. The studies described herein thus provides compelling evidence that CBD and analogs/derivatives thereof may be useful for the management of insomnia and/or sleep disorders associated to all types of chronic and/or neuropathic pain, and that CB1 antagonists may be useful for the management of disorders relating to REM sleep such as narcolepsy.

The present disclosure thus provides a method for the management of insomnia and/or sleep disorders associated to all types of chronic and/or neuropathic pain in a subject in need thereof comprising administering to the subject an effective amount of CBD or an analog thereof.

The present disclosure also provides the use of CBD or an analog thereof for the management of insomnia and/or sleep disorders associated to all types of chronic and/or neuropathic pain in a subject. The present disclosure also provides the use of CBD or an analog thereof for the manufacture of a medicament for the management of insomnia and/or sleep disorders associated to all types of chronic and/or neuropathic pain in a subject. The present disclosure also provides CBD or an analog thereof for use for the management of insomnia and/or sleep disorders associated to all types of chronic and/or neuropathic pain in a subject.

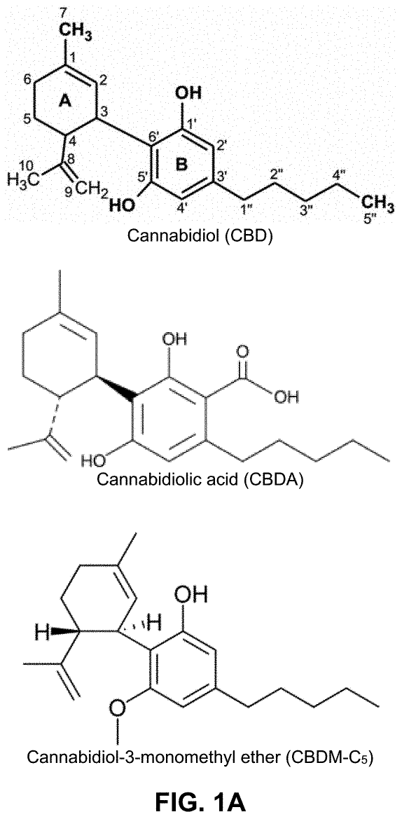

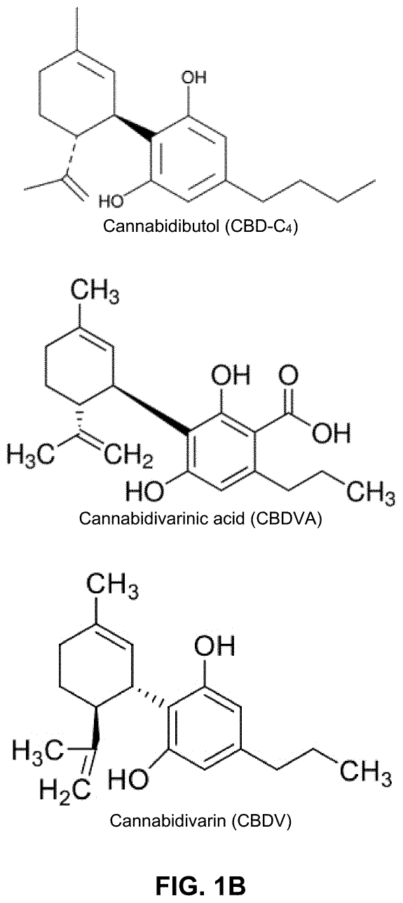

CBD is a terpenophenol compound containing twenty-one carbon atoms, with the formula C₂₁H₃₀O₂ and a molecular weight of 314.464 g/mol (FIG. 1A). The chemical structure of cannabidiol, 2-[1R-3-methyl-6R-(1-methylethenyl)-2-cyclohexen-1-yl]-5-pentyl-1,3-benzenediol, was determined in 1963²⁹. The current IUPAC preferred terminology is 2-[(1R,6R)-3-methyl-6-prop-1-en-2-ylcyclohex-2-en-1-yl]-5-pentylbenzene-1,3-diol. The CBD molecule contains a cyclohexene ring (A), a phenolic ring (B) and a pentyl side chain. In addition, the terpenic ring (A) and the aromatic ring (B) are located in planes that are almost perpendicular to each other³⁰. There are four known CBD side chain homologs, which are methyl, n-propyl, n-butyl, and n-pentyl³¹. All known CBD forms have absolute trans configuration in positions 1R and 6R³¹. The term CBD analog as used herein refers to a compound having the same backbone as CBD (e.g., a cyclohexene ring (A) linked to a phenolic ring (B)) and retaining the biological activity of CBD on pain-associated sleep disorders. Representative examples of CBD analogs include the compounds depicted in FIGS. 1A-1C. CBD analogs also includes pharmaceutically acceptable salts, solvates or prodrugs of CBD or of the CBD analogs depicted in FIGS. 1A-1C.

Provided here in some embodiments that a single or repeated (e.g., for multiple days) administration of oral formulation of CBD or CBD analogs exerts hypnotic and analgesic proprieties in patients under chronic and/or neuropathic pain condition (e.g. post herpetic (or post-shingles) neuralgia, reflex sympathetic dystrophy/causalgia (nerve trauma), cancer pain, phantom limb pain, entrapment neuropathy (e.g., carpal tunnel syndrome), and peripheral neuropathy (widespread nerve damage), diabetic neuropathy, lower back pain, pain induced by chemotherapy (cisplatin, paclitaxel, vincristine, etc.) or radiotherapy, HIV infection or AIDS, central nervous system disorders (stroke, Parkinson's disease, multiple sclerosis, etc.), complex regional pain syndrome, Nerve compression or infiltration by tumors.

In some embodiments, the oral or parental or topic formulation of CBD or CBD analogs will reduce insomnia and improve sleep quality in individuals under neuropathic pain condition (e.g. post herpetic (or post-shingles) neuralgia, reflex sympathetic dystrophy/causalgia (nerve trauma), components of cancer pain, phantom limb pain, entrapment neuropathy (e.g., carpal tunnel syndrome), and peripheral neuropathy (widespread nerve damage), diabetic neuropathy, lower back pain, pain induced by chemotherapy (cisplatin, paclitaxel, vincristine, etc.), or radiotherapy, HIV infection or AIDS, central nervous system disorders (stroke, Parkinson's disease, multiple sclerosis, etc.), complex regional pain syndrome, nerve compression or infiltration by tumors.

In certain embodiments, (e.g., pharmaceutical) compositions provided herein and/or methods provided herein are configured to provide a prolonged and/or repeated exposure in an individual to CBD or a CBD analog in a pharmaceutically acceptable salt, solvate, metabolite, derivative, or prodrug thereof.

In some embodiments, CBD in a pharmaceutically acceptable salt, solvate, metabolite, derivative, or prodrug thereof is provided to an individual at a first time point and a second time point. In certain embodiments, a composition provided herein comprises a first dosage form comprising a pharmaceutically acceptable salt, solvate, metabolite, derivative, or prodrug thereof, and a second dosage form comprising CBD in a pharmaceutically acceptable salt, solvate (the first and second dosage forms may be the same or different).

In some embodiments, the dosage form is administered acutely, subacutely or chronically. In some embodiments, additional dosage forms are also administered, such as on intervening days and/or subsequent to administration of the second dosage form.

In certain embodiments, a composition (or dosage form) provided herein is an immediate or a controlled (e.g., an extended) release composition (or dosage form)). In some instances, the immediate or controlled (e.g., an extended) release composition (or dosage form), or component thereof, provides exposure to CBD or CBD analog for an (e.g., extended) period of time. In some instances, the exposure is at or above a minimum effective (e.g., serum) concentration of CBD or CBD analog.

In some embodiments, the method or use is for managing insomnia associated to chronic and/or neuropathic pain condition, or the symptoms thereof in an individual in need thereof. In some embodiments, the individual is suffering from or susceptible to insomnia.

In some embodiments, the neuropathic pain condition is a peripheral neuropathy (widespread nerve damage). In some embodiments, the neuropathic pain condition is a post herpetic (or post-shingles) neuralgia. In some embodiments, the neuropathic pain condition is a reflex sympathetic dystrophy/causalgia (nerve trauma). In some embodiments, the neuropathic pain condition components of cancer pain. In some embodiments, the neuropathic pain condition phantom limb pain. In some embodiments, the neuropathic pain condition entrapment neuropathy (e.g., carpal tunnel syndrome). In some embodiments the neuropathic pain and/or chronic pain is diabetic neuropathy, lower back pain, pain induced by chemotherapy (cisplatin, paclitaxel, vincristine, etc.), or radiotherapy, HIV infection or AIDS, central nervous system disorders (stroke, Parkinson's disease, multiple sclerosis, etc.), complex regional pain syndrome, nerve compression or infiltration by tumors.

In some embodiments, individuals with neuropathic pain display difficulty falling asleep, maintaining sleep or waking up earlier.

In some embodiments, insomnia is a predominant complaint of dissatisfaction with sleep quantity or quality, associated with one (or more) of the following symptoms:

1. Difficulty initiating sleep. (In children, this may manifest as difficulty initiating sleep without caregiver intervention.)
2. Difficulty maintaining sleep, characterized by frequent awakenings or problems returning to sleep after awakenings. (In children, this may manifest as difficulty returning to sleep without caregiver intervention.)
3. Early-morning awakening with inability to return to sleep.

4. The sleep disturbance causes clinically significant distress or impairment in daytime functioning, as evidenced by at least one of the following:
Fatigue or low energy
Daytime sleepiness
Impaired attention, concentration, or memory
Mood disturbance
Behavioral difficulties
Impaired occupational or academic function
Impaired interpersonal or social function
Negative effect on caregiver or family functioning

The sleep difficulty occurs at least 3 nights per week, is present for at least 3 months.

The sleep difficulty occurs despite adequate opportunity for sleep³².

Sleep disorders can be here associated to a reduction of NREM sleep, and/or REM sleep and/or an increase time awakening at night and/or a non-restorative sleep and/or distress or impairment in social, occupational, educational, academic, behavioral, or other important areas of functioning due to a non-satisfactory sleep.

In an embodiment, the methods or uses described herein permit to (a) increase REM sleep time, (b) increase NREM sleep time, and/or (c) decrease wakefulness time in the neuropathic individual. In an embodiment, the methods or uses described herein permit to increase REM sleep time in the treated neuropathic individual. In an embodiment, the REM sleep time is increased by at least 25% or 50% relative to an untreated subject. In further embodiments, the REM sleep time is increased by at least 75%, 100% (2-fold) or 150% relative to an untreated subject. In an embodiment, the methods or uses described herein permit to increase NREM sleep time in the treated individual. In an embodiment, the NREM sleep time is increased by at least 10, 20 or 25% relative to an untreated subject. In further embodiments, the NREM sleep time is increased by at least 40 or 50% relative to an untreated subject. In an embodiment, the methods or uses described herein permit to decrease wakefulness time in the treated individual. In an embodiment, the wakefulness time is decreased by at least 10, 20 or 25% relative to an untreated subject. In further embodiments, the wakefulness time is decreased by at least 40 or 50% relative to an untreated subject.

In humans, NREM sleep is divided into three separate sub-stages: N1, N2 and N3 or slow-wave sleep (SWS). Thus, in certain embodiments, NREM sleep in rodents indicates also NREM or SWS in humans. SWS corresponds also to “restorative sleep” or “deep sleep”, helping the body to restore from fatigue, stress and diseases.

The present disclosure also provides a method for the management of a disorder associated with REM sleep (e.g., excessive REM sleep) in a subject comprising administering to the subject an effective amount of a CB₁ inhibitor. The present disclosure also provides the use of a CB₁ inhibitor for the management of a disorder associated with REM sleep (e.g., excessive REM sleep) in a subject, or for the manufacture of a medicament for the management of a disorder associated with REM sleep (e.g., excessive REM sleep) in a subject.

In an embodiment, the disorder associated with REM sleep is narcolepsy. In an embodiment, the narcolepsy is type 1 narcolepsy (previously referred to as narcolepsy with cataplexy). The diagnosis of type 1 narcolepsy is based on the individual either having low levels of a brain hormone (hypocretin) or reporting cataplexy and having excessive daytime sleepiness on a special nap test. In another embodiment, the narcolepsy is type 2 narcolepsy (previously referred to as narcolepsy without cataplexy). People with type 2 narcolepsy experience excessive daytime sleepiness but usually do not have muscle weakness triggered by emotions. They usually also have less severe symptoms than subjects with type 1 narcolepsy and have normal levels of the brain hormone hypocretin. Cataplexy refers to the sudden loss of muscle tone while a person is awake leads to weakness and a loss of voluntary muscle control and it is associated to REM episodes during the day; it is often triggered by sudden, strong emotions such as laughter, fear, anger, stress, or excitement.

In another embodiment, the disorder associated with REM sleep is a REM sleep behavior disorder (RBD). REM sleep behavior disorder are conditions characterized by sudden body movements and vocalizations while a person experiences vivid dreams during REM sleep. RBD is often associated with Alpha-synuclein neurodegeneration, as seen in Parkinson disease (PD), multiple system atrophy (MSA), and Lewy body dementia (also called dementia with Lewy bodies dementia, DLB). RBD also occurs in patients with Pontine lesions (structural lesions in the brainstem due to vascular, demyelinating, neoplastic, and traumatic etiologies), as well as in patients treated with a serotonergic antidepressant medication, such as selective serotonin reuptake inhibitors (SSRI), serotonin-norepinephrine reuptake inhibitors (SNRI), or serotonin modulating agents.

The term “CB₁ inhibitor” as used herein refers to an agent that is able to block or reverse the activation of the cannabinoid receptor 1 (CB₁), such as a CB₁ antagonist or a CB₁ inverse agonist. The CB₁ inhibitor may be a small molecule, a peptide, a polypeptide or an anti-CB₁ antibody. In an embodiment, the CB₁ inhibitor is a small molecule inverse agonist. In a further embodiment, the CB₁ inhibitor is Rimonabant (also known as SR141716) or an analog thereof. Analogs of Rimonabant include Surinabant (SR147778), AM1248 (1-((N-Methylpiperidin-2-yl)methyl)-3-(adamant-1-oyl)indole), NESS-0327 (8-Chloro-1-(2,4-dichlorophenyl)-1,4,5,6-tetrahydro-N-1-piperidinyl-benzo [6,7]cyclohepta[1,2-c]pyrazole-3-carboxamide), SLV-319 (ibipinabant), Otenabant (CP-945,598), taranabant (MK-0364), AVE1625 (N-[1-[Bis(4-chlorophenyl)methyl]-3-azetidinyl]-N-(3,5-difluorophenyl)methanesulfonamide), AM4113 (5-(4-Chlorophenyl)-1-(2,4-dichlorophenyl)-4-methyl-1H-pyrazole-3-carboxamide), AM6545 (5-[4-(4-Cyano-1-butyn-1-yl)phenyl]-1-(2,4-dichlorophenyl)-N-(1,1-dioxido-4-thiomorpholinyl)-4-methyl-1H-pyrazole-3-carboxamide), and AM251 (1-(2,4-dichlorophenyl)-5-(4-iodophenyl)-4-methyl-N-(piperidin-1-yl)-1H-pyrazole-3-carboxamide). Example of anti-CB₁ antibodies include namacizumab, brizantin and Dietressa.

In an embodiment, the CBD or CBD analog, or CB₁ inhibitor, is formulated into a pharmaceutical composition that comprises at least one pharmaceutically acceptable carrier or excipient. Such compositions may be prepared in a manner well known in the pharmaceutical art art by mixing the active ingredient (CBD or CBD analog, or CB₁ inhibitor) having the desired degree of purity with one or more optional pharmaceutically acceptable carriers, excipients and/or stabilizers (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). Supplementary active compounds can also be incorporated into the compositions. The carrier/excipient can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration.

An “excipient,” as used herein, has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive.

Useful diluents, e.g., fillers, include, for example and without limitation, dicalcium phosphate, calcium diphosphate, calcium carbonate, calcium sulfate, lactose, cellulose, kaolin, sodium chloride, starches, powdered sugar, colloidal silicon dioxide, titanium oxide, alumina, talc, colloidal silica, microcrystalline cellulose, silicified micro crystalline cellulose and combinations thereof. Fillers that can add bulk to tablets with minimal drug dosage to produce tablets of adequate size and weight include croscarmellose sodium NF/EP (e.g., Ac-Di-Sol); anhydrous lactose NF/EP (e.g., Pharmatose™ DCL 21); and/or povidone USP/EP.

Binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Sil™ M5P), Silicified Microcrystalline Cellulose (SMCC), e.g., Silicified microcrystalline cellulose NF/EP (e.g., Prosolv™ SMCC 90), and silicon dioxide, mixtures thereof, and the like), veegum, and combinations thereof.

Useful lubricants include, for example, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), dl-leucine, calcium stearate, sodium stearyl fumarate, mixtures thereof, and the like.

Bulking agents include, for example: microcrystalline cellulose, for example, AVICEL® (FMC Corp.) or EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX®).

Disintegrating or dissolution promoting agents include: starches, clays, celluloses, alginates, gums, crosslinked polymers, colloidal silicon dioxide, osmogens, mixtures thereof, and the like, such as crosslinked sodium carboxymethyl cellulose (AC-DI-SOL®), sodium croscarmelose, sodium starch glycolate (EXPLOTAB®, PRIMO JEL®) crosslinked polyvinylpolypyrrolidone (PLASONE-XL®), sodium chloride, sucrose, lactose and mannitol.

Antiadherents and glidants employable in the core and/or a coating of the solid oral dosage form may include talc, starches (e.g., cornstarch), celluloses, silicon dioxide, sodium lauryl sulfate, colloidal silica dioxide, and metallic stearates, among others.

Examples of silica flow conditioners include colloidal silicon dioxide, magnesium aluminum silicate and guar gum.

Suitable surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, flavoring, coloring and/or sweetening agents may be added as well.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

Examples of thickening agent can be for example talc USP/EP, a natural gum, such as guar gum or gum arabic, or a cellulose derivative such as microcrystalline cellulose NF/EP (e.g., Avicel™ PH 102), methylcellulose, ethylcellulose or hydroxyethylcellulose. A useful thickening agent is hydroxypropyl methylcellulose, an adjuvant which is available in various viscosity grades.

Examples of plasticizers include: acetylated monoglycerides; these can be used as food additives; Alkyl citrates, used in food packagings, medical products, cosmetics and children toys; Triethyl citrate (TEC); Acetyl triethyl citrate (ATEC), higher boiling point and lower volatility than TEC; Tributyl citrate (TBC); Acetyl tributyl citrate (ATBC), compatible with PVC and vinyl chloride copolymers; Trioctyl citrate (TOC), also used for gums and controlled release medicines; Acetyl trioctyl citrate (ATOC), also used for printing ink; Trihexyl citrate (THC), compatible with PVC, also used for controlled release medicines; Acetyl trihexyl citrate (ATHC), compatible with PVC; Butyryl trihexyl citrate (BTHC, trihexyl o-butyryl citrate), compatible with PVC; Trimethyl citrate (TMC), compatible with PVC; alkyl sulphonic acid phenyl ester, polyethylene glycol (PEG) or any combination thereof. Optionally, the plasticizer can comprise triethyl citrate NF/EP.

Examples of permeation enhancers include: sulphoxides (such as dimethylsulphoxide, DMSO), azones (e.g., laurocapram), pyrrolidones (for example 2-pyrrolidone, 2P), alcohols and alkanols (ethanol, or decanol), glycols (for example propylene glycol and polyethylene glycol), surfactants and terpenes.

Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds/compositions of the invention include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

In an embodiment, the pharmaceutical composition is a delayed-release formulation or dosage form. Delayed release can be effected by the use of one or more release-delaying agents. Any combination of release-delaying agents, including the ones described herein, can be used in the dosage forms. The release-delaying agent acts to increase the period before release begins from a dosage form. The length of the lag period before delayed release occurs can by controlled using methods known to those of skill in the art, for instance by varying the choice, combination, form, shape and/or amount of release-delaying agent(s).

The delayed release formulations can be prepared, for example, by coating active ingredient or an active ingredient-containing composition with one or more release-delaying agents. In other instances, the release-delaying agent(s) can be intermixed with or in co-solution with the active ingredient. For example, delayed release by osmotic rupture can be achieved by a dosage form comprising one or more swelling agents that are contained in combination with the active ingredient within a semipermeable coating. The increase in volume of the swelling agent upon exposure of the unit dosage form to bodily fluids causes the semipermeable coating to rupture. In such agents, both the swelling agent and the semipermeable coating can be considered to be release-delaying agents. Thus, delayed release can be achieved by a combination of release-delaying agents, where each release-delaying agent does not necessarily delay release by itself.

Delayed release can be achieved by various processes such as dissolution, diffusion, erosion (e.g., based on the inherent dissolution of the agent and incorporated excipients), and/or rupture (e.g., by swelling). Common mechanisms include bulk erosion of polymers which restrict diffusion of the drug, surface erosion, (e.g., of layered medicaments), or rupture. Rupture can be osmotically controlled, for instance by swelling that results from the osmotic infusion of moisture.

Rupture can also result from the reaction of effervescent agents, e.g., citric acid/sodium bicarbonate, with water or other fluids that penetrate into the dosage form. Release, including delayed release, from a unit dosage form can be achieved by more than one mechanism. For example, release can occur for example by simultaneous swelling and diffusion, simultaneous diffusion and erosion, and simultaneous swelling, diffusion and erosion.

Methods of making delayed-burst release formulations are within ordinary skill. Examples are presented herein and can also be found in numerous publications, including.

Alternatively, delayed release can be initiated by a triggering signal such as a fluctuation in temperature, or an electromagnetic pulse. See, e.g., US Patent Publication Nos. 2001/6251365, 2006/997863, 2003/6514481, 2006/0057737, 2006/0178655, 2006/0121486, and 2006/0100608.

Two common classes of release-delaying agents are “enteric” (allowing release within a specific milieu of the gastro-intestinal tract) and “fixed-time” (allowing release after a “predetermined” or “fixed” time period after administration, regardless of gastro-intestinal milieu), each of which is discussed in more detail below. Enteric release-delaying agents for instance allow release at certain pHs or in the presence of degradative enzymes that are characteristically present in specific locations of the GI tract where release is desired. The formulation may comprise more than one release-delaying agent from any class, such as a combination of enteric and fixed-time release-delaying agents. In another embodiment, the release-delaying agent allows the release of drug after a predetermined period after the composition is brought into contact with body fluids (“fixed-time delayed release”). Unlike enteric release, fixed-time release is not particularly affected by environmental pH or enzymes.

A large number of fixed-time release-delaying agents are known to those of ordinary skill in the art. Exemplary materials which are useful for making the time-release coating of the invention include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose, cellulose acetate, cellulose acetate butyrate and ethyl cellulose; and other materials known to those of ordinary skill in the art. Other film-forming materials that can be used for this purpose include poly(vinylpyrrolidone), polyvinylalcohol, polyethylene oxide, a blend of gelatin and polyvinylpyrrolidone, gelatin, glucose, saccharides, povidone, copovidone, poly(vinylpyrrolidone)-poly(vinyl acetate) copolymer. Other materials which can be used in the time-release coating include Acryl-EZE®, Eudragit® NE, RL and RS, hydroxypropylcellulose, microcrystalline cellulose (MCC, Avicel™ from FMC Corp.), poly(ethylene-vinyl acetate) (60:40) copolymer (EVAC from Aldrich Chemical Co.), 2-hydroxyethylmethacrylate (HEMA), MMA, and calcium pectinate can be included. Substances that are used as excipients within the pharmaceutical industry can also act as release-delaying agents.

Common types of fixed-time release dosage forms include erodible formulations, formulations that undergo osmotic rupture, or unit dosage form that use any combination of mechanisms for delayed release.

Fixed-time release-delaying agents can optionally achieve a delayed-burst release by osmotic rupture. Examples of such RDAs include swelling agents, osmogens, binders, lubricants, film formers, pore formers, coating polymers and/or plasticizers.

Osmotic rupture is achieved by a delayed release component which comprises a coated unit dosage form that contains the drug and a swelling agent within the semipermeable coating (e.g., ethylcellulose). The coating weight (thickness) of the semipermeable coating can be selected to delay release by osmotic rupture for a desired period. To identify the correct coating weight for a particular delay, unit dosage forms with a range of coating weights can be tested via in vitro dissolution to determine the burst time. Based on these results, a coating weight that achieves the desired lag period would be selected. In addition, the amount and/or ratio of a coating strength modifier (e.g., talc) in the coating can be adjusted as well. Other formulation variables that can also be adjusted to obtain the desired release by osmotic rupture include the amount of sweller layer and sweller and/or fillers in the formulation. In the case of rupturing tablets, the amount of sweller would be selected to achieve the target release, while still providing the tablet with sufficient compressibility and acceptably low friability to be manufacturable.

In an embodiment, the formulation may comprise one or more “diffusion regulators” that control the permeation of bodily fluids into the drug-containing core. Exemplary diffusion regulators include hydrophilic polymers, electrolytes, proteins, peptides, amino acids and others known to those of ordinary skill in the pharmaceutical sciences. In an example, the fixed-time release-delaying agent comprises a coating that permits release of the active ingredient after a fixed period. The thickness of the coating can affect the time required for penetration of fluids into the formulation. For example, and without limitation, a diffusion controlling time release coating that provides release after a fixed delay period of about 0.5-2.5 hours could be about 200-1000 microns thick, and one that provides a release after a fixed delay period of about 2.5-5.0 hours could be about 1000-3000 microns thick.

Erodible formulations provide another example of fixed-time release formulations. The release delay from an erodible coated tablet can be adjusted by those of ordinary skill in the art by regulating the erodible layer coating weight. To identify the correct coating weight, tablets over a range of coating weights can be tested via in vitro dissolution (and/or erosion) to determine the burst time. Other formulation variables that may affect performance include the selection of the coating layer polymer type and viscosity. In an embodiment, the formulation may comprise one or more “erosion regulators” that control the erosion rate of the coating. Any material or combination of materials may serve as an erosion regulator. Exemplary erosion and/or diffusion regulators include hydrophilic polymers, electrolytes, proteins, peptides, amino acids and others known to those of ordinary skill in the pharmaceutical sciences. The thickness of the coating can affect the time required for erosion of the coating. For example, and not limitation, an erodible time-release coating that provides release after a fixed period of about 0.5-2.5 hours could be about 100-2000 microns thick, and one that provides release after a fixed delay period of about 2.5-5.0 hours could be about 2000-5000 microns thick.

The release-delaying agent may comprise an “enteric” material that is designed to allow release upon exposure to a characteristic aspect of the gastrointestinal tract. In an embodiment, the enteric material is pH-sensitive and is affected by changes in pH encountered within the gastrointestinal tract (pH-sensitive release). The enteric material typically remains insoluble at gastric pH, then allows for release of the active ingredient in the higher pH environment of the downstream gastrointestinal tract (e.g., often the duodenum, or sometimes the colon). In another embodiment, the enteric material comprises enzymatically degradable polymers that are degraded by bacterial enzymes present in the lower gastrointestinal tract, particularly in the colon. Optionally, the unit dosage form is formulated with a pH-sensitive enteric material designed to result in a release within about 0-2 hours when at or above a specific pH. In various embodiments, the specific pH can for example be from about 4 to about 7, such as about 4.5, 5, 5.5, 6, 6.5 or 7.

Materials used for enteric release formulations, for example as coatings, are well known in the art and include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the trade-name Acryl-EZE® (Colorcon, USA), Eudragit® (Rohm Pharma; Westerstadt, Germany), including Eudragit® L30D-55 and L100-55 (soluble at pH 5.5 and above), Eudragit® L-IOO (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different enteric materials may also be used. Multi-layer coatings using different polymers may also be applied. The properties, manufacture and design of enteric delivery systems are well known to those of ordinary skill in the art. See, e.g., Development of Biopharmaceutical Parenteral Dosage Forms (Drugs and the Pharmaceutical Sciences), by Bontempo (Publishers: Informa Healthcare (Jul. 25, 1997).

Those of ordinary skill in the art can adjust the period before delayed release from enteric coated multiparticulates by varying the enteric layer coating weight and composition. For example, where time in the stomach is <4 hours and some amount of protection (1-3 hours) is desired after the dosage form leaves the stomach, then an appropriate level of coating that provides up to 4 hours of protection between administration and drug release can be prepared.

The release interval can be determined in vitro or in vivo. Although the plasma concentration of a drug can lag behind the actual time of release in the GI tract, the release interval can be approximately determined in vivo as the time interval between the Cₘₐₓ (i.e., the maximum plasma concentration) of the active ingredients achieved by the immediate release component and the Cₘₐₓ of the active ingredients achieved by the delayed release component. Alternatively, the release interval can be monitored through the increased plasma concentration of the active ingredients caused by delayed release following immediate release, compared to that achieved by only the immediate release of the active ingredients.

Release can also be assessed using commonly used in vitro dissolution assays. Generally, an in vitro dissolution assay is carried out by placing the dosage form(s) (e.g., tablet(s)) in a known volume of dissolution medium in a container with a suitable stirring device. An aliquot of the medium is withdrawn at various times and analyzed for dissolved active substance to determine the rate of dissolution. In one approach, the dosage form (e.g., tablet) is placed into a vessel of a United States Pharmacopeia dissolution apparatus II (Paddles) containing 900 ml dissolution medium at 37° C. The paddle speed is 50, 75 or 100 RPM. Independent measurements are made for at least three (3) tablets, e.g., 6 tablets. The dissolution medium can be a neutral dissolution medium such as 50 mM potassium phosphate buffer, pH 7.2 (“neutral conditions”) or water or an acidic medium such as 50 mM potassium (or sodium) acetate buffer, at pH 4.5. Typically, a unit dose form is added to the vessel and dissolution is started. At specified times, e.g., 5, 10, 15, 20, 30, 45 or 60 minutes, an aliquot (e.g., 2 ml) of medium is withdrawn and the amount of active ingredient in solution is determined using routine analytical methods (e.g., HPLC).

By way of example, immediate release and/or delayed release of drugs from the dosage form can be monitored using Apparatus II (Paddles) as described in U.S. Pharmacopeia, where the dissolution is conducted by placing one dosage form into each of six vessels containing 900 ml of release media with temperature at 37° C. and speed of 100 rpm. Optionally, the release media of 0.1N Hydrochloric acid (pH 1.2 or 4.5) is used for stage 1 for 2 hours, and 0.2M tribasic sodium phosphate buffer adjusted to pH6.8 is used for stage 2 (Buffer stage) at 5, 10,15, 20, 30, 45, 60, 90 and 120 minutes and assayed for drug content by HPLC. Further, various media for in vitro dissolution assays (e.g., simulated gastric fluid (SGF), simulated intestinal fluid (SIF), versions to simulate fed or fasting conditions (FeSSGF or FeSSIF for fed conditions, FaSSGF or FaSSIF for fasting conditions), etc.) are well known in the art.

In an embodiment, the CBD or analog thereof is administered or is for administration to the human subject via parenteral administration (i.e., intramuscular, subcutaneous, intravenous, or intradermal) at a dose of about 1 or 2 mg/kg to about 25 or 30 mg/kg, preferably about 5 mg/kg to about 15 or 20 mg/kg.

In an embodiment, the CB₁ inhibitor is administered or is for administration to the human subject via parenteral administration (i.e., intramuscular, subcutaneous, intravenous, or intradermal) at a dose of about 0.05 or 0.1 mg/kg to about 5 or 10 mg/kg, preferably about 0.1 mg/kg to about 5 mg/kg, for example about 0.5 to about 2 mg/kg or about 1 mg/kg.

In an embodiment, the CBD or analog thereof is administered or is for administration to the human subject at a dose corresponding to a dose of about 1 or 2 mg/kg to about 25 or 30 mg/kg, preferably about 5 mg/kg to about 15 or 20 mg/kg, in rats. Based on the equivalence factor of 0.162 between dosages in humans and rats (see, e.g., Nair and Jacob, J Basic Clin Pharm. 2016 March; 7(2):27-31), a dose of about 5 mg/kg to about 20 mg/kg in rats corresponds to a dose of about 0.8 mg/kg (5*0.162) to about 3.25 mg/kg (20*0.162) in human subjects. In a further embodiment, the CBD or analog thereof is administered or is for administration to the human subject at a dose of about 3 to about 3.5 mg/kg, about 3.1 to about 3.4 mg/kg, or about 3.2 to about 3.3 mg/kg. In an embodiment, the administration is via parenteral administration (i.e., intramuscular, subcutaneous, intravenous, or intradermal).

In an embodiment, the CB₁ inhibitor is administered or is for administration to the human subject at a dose corresponding to a dose of about 0.05 or 0.1 mg/kg to about 5 or 10 mg/kg, preferably about 0.1 mg/kg to about 5 mg/kg, for example about 0.5 to about 2 mg/kg or about 1 mg/kg, in rats.

In another embodiment, the CBD or analog thereof is administered or is for administration per os (oral administration). In an embodiment, in view of the low bioavailability of CBD and analogs thereof, the CBD or analog thereof is administered or is for administration per os (oral administration) at a daily dosage of about 50 mg to about 1000, 900 or 800 mg, about 50 mg to about 700 mg, about 50 mg to about 600 mg, about 50 mg to about 500 mg, about 50 mg to about 400 mg, about 75 mg to about 300 mg, or about 100 mg to about 300 mg. In further embodiment, the CBD or analog thereof is administered or is for administration per os at a daily dosage of about 50, 75, 100, 125, 150, 175, 200, 225, 250, 275, 300, 325, 350, 375 or 400 mg.

In an embodiment, the dose of CBD or analog thereof is administered or is for administration once or twice a day.

### EXAMPLES

The present disclosure is illustrated in further details by the following non-limiting examples.

### Example 1: Materials and Methods

Animals. The experiments were performed on male Wistar rats weighing 250 g (six weeks). All animals were housed in standardized animal facilities under a 12 h light/dark cycle (lights on at 7 AM) with ad libitum access to food and water. All surgeries and experimental procedures were performed during the light cycle. Experimental protocols were approved by the Animal Ethics Committee of the local institutional committee for animal use and care (McGill University, Qc, Canada). These protocols follow ethical guidelines for investigation of experimental pain in conscious animals of the IASP, the Canadian Institute of Health Research guidelines for animal care and scientific use.

Drug. Cannabidiol (Cayman Chemical, Ann Arbor, Mich.) was prepared in a vehicle of ethanol/Tween® 80/0.9% saline (3:1:16). The 5-HT1A antagonist WAY 100635 (Tocris Bioscience, Ellisville, Mo.) was dissolved in 0.9% saline. The CB1 antagonist rimonabant or SR S141716A (CBD; Tocris Bioscience, Ellisville, Mo.) was prepared in a vehicle of PEG 400/Tween® 80/0.9% Saline (1:1:18).

Spared nerve injury (SNI). SNI was performed according to the method of Decosterd and Woolf³⁵. Rats were deeply anaesthetized with isofluorane (5%), anaesthesia was confirmed by the absence of a nociceptive reflex reaction to a paw pinch. The sciatic nerve was exposed at mid-thigh level distal to the trifurcation and freed of connective tissue; the three peripheral branches (sural, common peroneal, and tibial nerves) of the sciatic nerve were exposed without stretching nerve structures. Both tibial and common peroneal nerves were ligated and transected together. Carprofen (5 mg/kg) was administrated subcutaneously pre-surgery as well as three days every 24 hours post-surgery as analgesic. After SNI, each rat was housed in its home cage for 15 days until the neuropathy is developed. Naïve rats did not undergo any surgery^{36, 37}.

Mechanical allodynia. On day 15 after SNI, mechanical allodynia was assessed via von Frey test³⁸. Rats were placed in a test chamber, and they were allowed to acclimate for 60 minutes. Tactile allodynia was determined by measuring paw withdrawal thresholds in response to mid-plantar hind paw stimuli with Calibrated von Frey filaments³⁶. These filaments are of a logarithmically incremental stiffness corresponding to an applied force ranging from 0.4 to 15 g. Every filament was applied during 10 seconds in the hind paw to measure the withdrawal threshold. The 2.0 g force filament was applied first; in the presence of a response, the next smaller filament was applied. In the absence of a response, the next higher filament was applied. After the first change in response, the test continued until six responses were collected. The paw withdrawal threshold was then converted to the cutaneous nociceptive threshold by using the “up-down” method³⁹. The stimulus intensity (filament stiffness) required to produce a response in 50% of the applications for each animal was defined as 50% withdrawal threshold (expressed in g). The 50% withdrawal threshold was determined according to the following equation: 50% Threshold (g)=(10\hat{ } [Xf+kδ])/10,000. Where Xf is the value of the last von Frey filament used (in logarithmic units), k is the correction factor based on the response patterns of a calibration table and the tabulated value based on the pattern of positive and negative responses, and δ indicates the average differences between stimuli in logarithmic units^{38,39}. In non-lesioned animals (naïve) a value 11.98-15 g was considered normal, while the presence of allodynia was considered when the 50% withdrawal threshold of the limb is less than 4 g. All nerve-ligated rats were verified to be allodynic before the experiments. Rats without allodynia were excluded.

EEG/EMG Implantation. Neuropathic rats (10 days after surgery) and naïve rats were placed in a stereotaxic frame following isoflurane-induced anesthesia (5%). Anaesthesia was confirmed by the absence of a nociceptive reflex reaction to a paw pinch. For EEG monitoring, three stainless-steel epidural electrodes were positioned through 1.5 mm burr holes: one over the parietal cortex on each side, and the third (as a reference) in the right prefrontal cortex. In rats, their respective locations relative to bregma were −2 mm anteroposterior (AP) and −3 mm lateral (L), −7 mm AP and −3 mm L, and −4.5 mm AP and +3 mm L, according to Paxinos and Watson. For EMG monitoring, three flexible stainless-steel wire electrodes, silicon insulation removed at the terminal 3-4 mm, were implanted into the neck muscles (two bilaterally and one in the middle). Wires were fused with the electrodes and the connectors were fixed to the skull with dental acrylic (Coltene/Whaledent). Carprofen (10mg/kg) was administrated subcutaneously pre-surgery as well as three days every 24 hours post-surgically as analgesic. Each rat was single-caged post-surgery and allowed to recover for the next 5 days¹³. 24 hours after surgery, the rats were placed in the recording chamber and connected to a flexible 6-flat wire (3M Scotchflex™) in a freely moving manner for several hours during the subsequent 5 days. No recordings were performed, but tolerance to the cable and sleep behavior were observed. All rats from a home cage were also grouped for one hour each day for socialization to circumvent anxious-depressive-like behavior induced by social isolation¹³.

Study design for EEG and EMG assessment. For EEG and EMG recordings, CBD's vehicle [ethanol/Tween® 80/0.9% saline (3:1:16)], THC (5 mg/kg), CBD (20 mg/kg), WAY (2 mg/kg), and rimonabant (1 mg/kg) were injected via intraperitoneal administration at the beginning of the recording period (6 A.M.). To investigate the participation of CB1 and 5-HT1A receptors in the hypnotic effect of CBD, WAY (2 mg/kg) were injected 10 min before an effective dose of CBD (20 mg/kg) . In another cohort of rats rimonabant (1 mg/kg) was also injected i.p. at 6 A.M. in neuropathic rats.

EEG and EMG recording. Six days after surgery (16 days post-SNI), mechanical allodynia was assessed via von Frey test, and EEG/EMG was recorded for a period of 6 h (from 6 AM to 12 PM). The treatments (described below) were performed at 6:00 A.M., right after the recording had started. EEG/EMG signals were amplified at a total gain of 10.000 and filtered locally (EEG, low filter, 1 Hz; high, 1 kHz; EMG, low filter, 30 Hz; high, 3 kHz; Grass, P55), digitized using a CED power 1401 converter and Spike 2 software (CED), stored with a resolution of 128 Hz, and displayed on a PC monitor. Consecutive 10 s epochs were subjected to a fast Fourier transform (FFT), and EEG power spectra density was computed in the frequency range of 0-64 Hz¹³.

Analysis of EEG and EMG data. The three classical vigilance states as described in the rat were discriminated on the basis of the cortical EEG and neck EMG activities. Wakefulness was identified by a low-amplitude and desynchronized EEG signal (alpha waves 8-13 Hz), with sustained EMG activity (8-13 Hz). NREM sleep was clearly distinguished by high-voltage delta waves (1-4 Hz) and spindles (10-15 Hz) associated with a weak EMG activity. REM sleep was characterized by a low-amplitude EEG, comparable to that of wakefulness, with a pronounced theta rhythm (4-8 Hz) and a complete loss of muscle tone. Thresholds of EEG and EMG signals between NREM and REM sleep were kept consistent within each animal across the recording period. To avoid transitional periods such as drowsiness, only periods of typical stationary EEG and EMG lasting at least 10 s were considered for further analyses of wakefulness, NREMS, and REMS¹³.

To determine the variations in power spectra induced by the pharmacological treatment of CBD and antagonists, the power densities were summed up over the frequency band of 0-25 Hz (total power). The data were then standardized by expressing all power spectral densities at the different 0.5 Hz bin frequency ranges (delta, theta, and sigma) as a percentage relative to the total power of the same epoch¹⁶. Frequency ranges were 0-3.5Hz for delta, 4-8.5Hz for theta, and 11-14 Hz for sigma bands. The sum of power densities over a frequency band following treatments were expressed as the percentage difference from that in or naïve+veh or NP+vehicle group in statistical analysis. In the experiments with WAY 100635 the NP+CBD was compared with NP+vehicle, NP+WAY, NP+WAY+CBD.

Sleep fragmentation analysis. Sleep Fragmentation Index (SFI) is determinant of excessive daytime sleepiness as it is characterized by repetitive short interruptions of sleep by periods of wakefulness. SFI is significantly related to indices of sleep discontinuity and is correlated with number of microarousal index (MAI)⁴⁰. In this study, SFI is calculated as the total number of wakefulness episodes in 24 h divided by the total sleep time in hours during 24 h (REM+NREM).

Study design for EEG and EMG assessment. For EEG and EMG recordings, CBD's vehicle [ethanol/Tween® 80/0.9% saline (3:1:16)], (20 mg/kg), were injected via intraperitoneal administration at the beginning of the recording period (6 A.M).

Statistical Analysis. Data were analyzed with GraphPad Prism version 8.1.1 (Graph-Pad Software). Results are expressed as the mean±SEM. Two-way mixed-design ANOVA was used to analyze statistical differences in mechanical allodynia, using treatment, and time as factors in the analysis. One-way ANOVA was used to analyze statistical differences in the area under the curve, using treatment as factor in the analysis. Unpaired t-test (one-tailed) was used to compare statistical differences between naïve rats and neuropathic rats in EEG/EMG recordings. One-way ANOVA was used to calculate statistical differences between groups in EEG/EMG recordings, using treatment as factor in the analysis. One-way ANOVA was used to analyze statistical differences in number of awakenings, REM and NREM sleep events, using treatment as factor in the analysis. Two-way mixed-design ANOVA was used to analyze statistical differences between groups in in vivo electrophysiological recordings, using treatment, and time as factors in the analysis. Bonferroni post hoc tests were used to calculate statistical differences between groups. Only p values <0.05 were considered significant.

### Example 2: Increasing Doses of CBD Produce Anti-Allodynic Effects in Neuropathic Rats

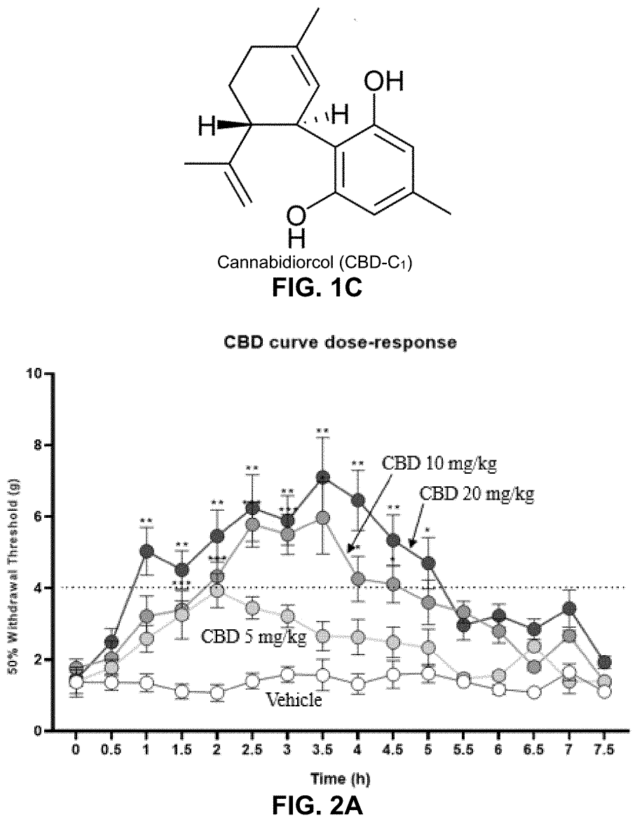

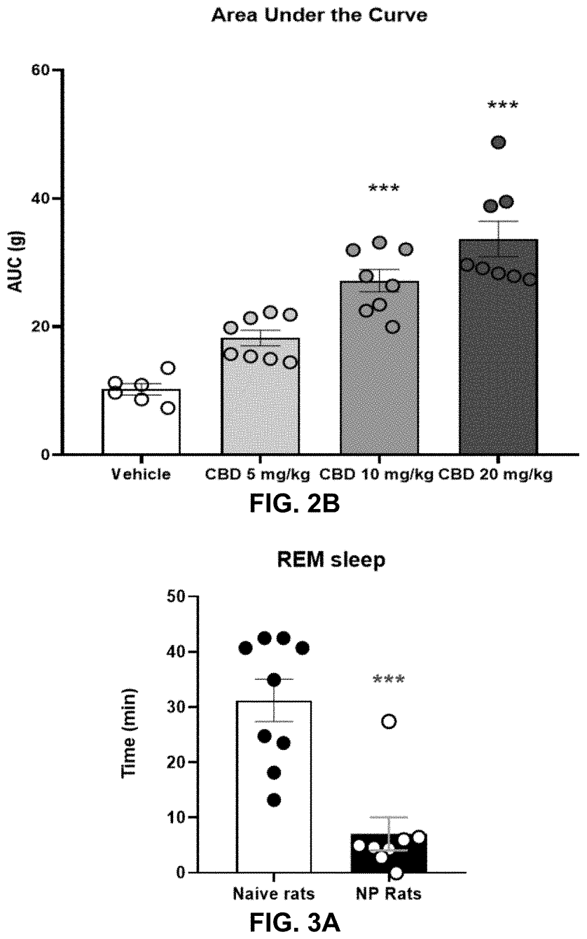

The intraperitoneal administration of CBD was able to reverse tactile allodynia induced by the SNI, increasing the paw withdrawal threshold in a dose-dependent manner, whereas administration of the vehicle did not change the withdrawal threshold (FIG. 2A). Two-way, mixed-design, ANOVA on the time course of CBD at different doses revealed a significant interaction between treatment and time (F_{[45, 390]}=3.545; p<0.001). Bonferroni post hoc comparisons computed on the simple main effect of dose over hours revealed that CBD treatment was able to reverse mechanical allodynia at the dose of 20 mg/kg between 1 h and 5 h (p<0.05), and at the dose of 10 mg/kg between 2 h and 4.5 h (p<0.05). Whereas CBD at 5 mg/kg did not show any antiallodynic effect. These effects lasted up to 4 hours post-administration (FIG. 2A). A one factor between-subject ANOVA performed on the AUC revealed a significant effect for group (F_{[3, 26]}=27.22, p<0.001; FIG. 2B). Subsequent Bonferroni post hoc analysis indicated that the AUC of the 20 mg/kg group was significantly higher compared to the AUC of the group receiving CBD at 5 mg/kg (p<0.001) and to the group receiving vehicle (p<0.001). Even though the AUC of the doses of 10 and 20 mg/kg were not statistically different, the 20 mg/kg dose of CBD was used for the following experiments.

### Example 3: Neuropathic Animals Develop Electrophysiological Sleep Perturbation/Fragmentation

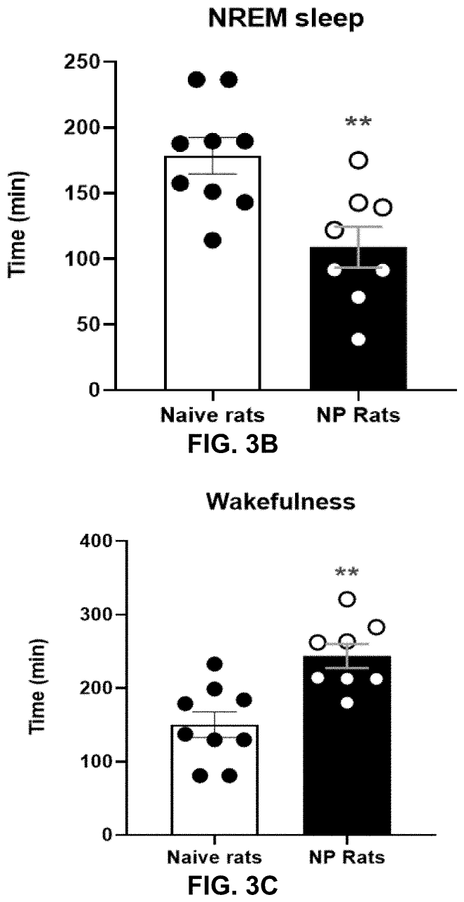

To assess whether neuropathy might induce sleep fragmentation also in rats, EEG/EMG parameters (wakefulness, NREM and REM sleep) were measured for 6 hours in NP and naïve rats. An unpaired t-test (one-tailed) conducted on data revealed that the mean wakefulness for NP rats (n=11) was significantly higher than the mean for naïve rats (n=9), t[18]=3.007, p<0.01 (FIG. 3C). An unpaired t-test (one-tailed) revealed the average spent in NREM sleep by NP rats (FIG. 3B) was significantly lower compared to that of naïve rats, t[18]=2.912, p<0.01. In addition, also the average of time spent in REM sleep by NP rats was statistically lower when compared to that of naïve rats, t[18]=3.862, p<0.001 (one-tailed; FIG. 3A). Therefore, sleep fragmentation may be used as a biomarker of neuropathic pain.

### Example 4: CBD Reverses the Sleep Perturbations Induced by Neuropathic Pain

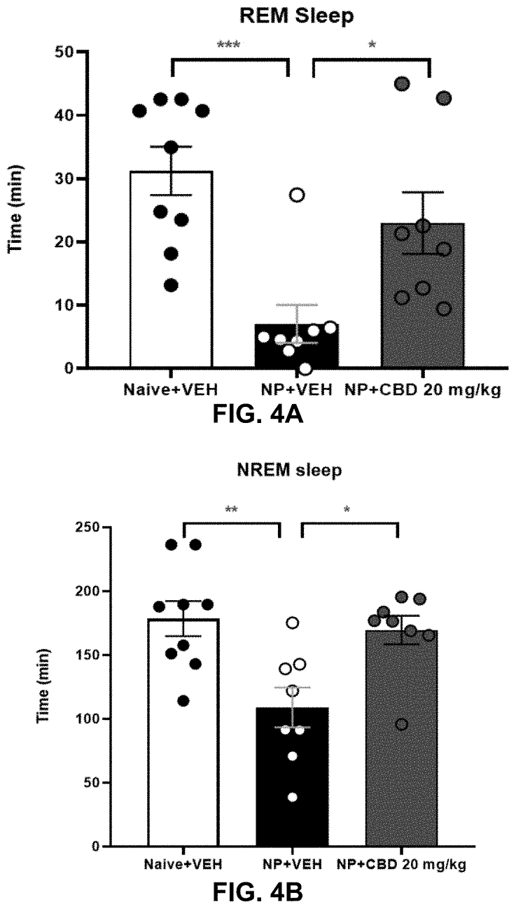

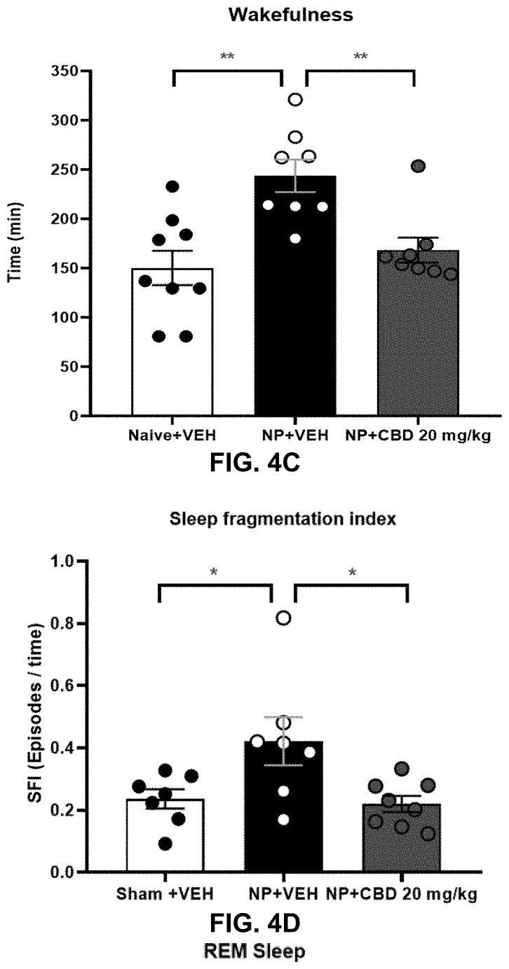

To assess whether CBD (20 mg/kg) might reverse sleep fragmentation associated with NP, EEG/EMG parameters (wakefulness, NREM and REM sleep) were measured for 6 hours in three groups of rats. The first group were naïve rats treated with CBD vehicle (n=9), the second group were NP rats treated with CBD vehicle (n=8), and the third group were NP rats treated with a CBD dose of 20 mg/kg (n=8). One-way ANOVA performed on the total time of wakefulness (F_{[2, 22]}=9.888, p<0.001) revealed a statistically significant difference between groups (FIG. 4C). Bonferroni post-hoc analysis revealed that NP rats treated with CBD display a decrease in the amount of time spent in wakefulness, relative to NP rats treated with vehicle (p<0.01), whereas no difference was detected between naïve animals treated with vehicle and NP rats treated with CBD. One-way ANOVA between subjects performed on total NREM sleep revealed a significant main effect for groups (F_{[2, 22]}=7.553, p<0.01). Bonferroni post-hoc comparison revealed that CBD (20 mg/kg) was able to reverse NREM sleep impairments in NP rats (p<0.05, FIG. 4B) relative to NP rats treated with vehicle, whereas no difference was detected between naïve animals treated with vehicle and NP rats treated with CBD. Additionally, one-way ANOVA between subjects performed on REM sleep (total time) revealed a significant main effect for groups (F_{[2,22]}=9.680, p<0.001, FIG. 4A). Bonferroni post-hoc analysis showed CBD (20 mg/kg) administration restored the normal REM sleep in neuropathic rats (p<0.05) relative to NP rats treated with vehicle and no difference was detected between naïve animals treated with vehicle and NP rats treated with CBD. The results depicted in FIG. 4D show that CBD attenuates the sleep fragmentation (repetitive short interruption of sleep) induced by neuropathy, as shown by a decrease of the sleep fragment index (SFI) to normal levels in neuropathic animals treated with CBD.

### Example 5: The 5-HT1A Antagonist WAY100635 Blocks the Hypnotic Effects of CBD

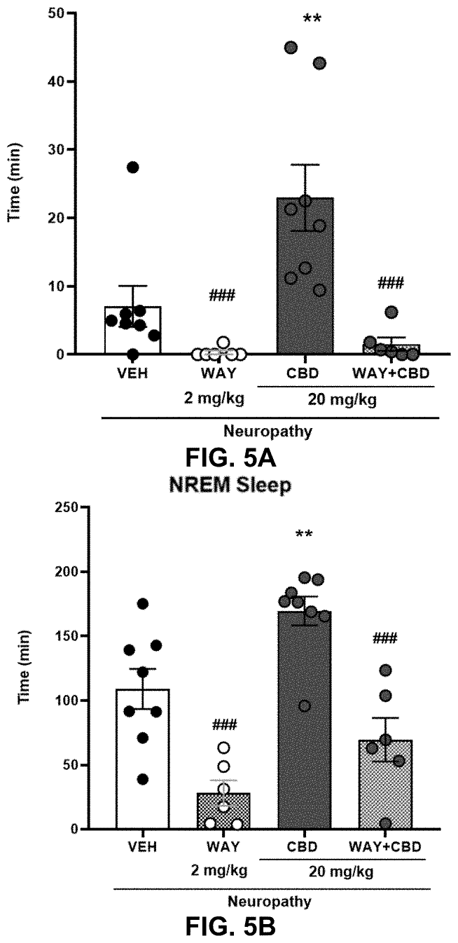

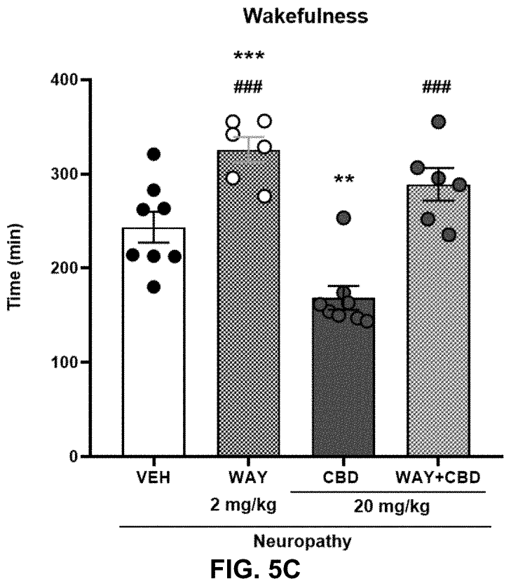

To assess whether the 5-HT1A selective antagonist WAY 100635 (2 mg/kg) might reverse the hypnotic effect of CBD (20 mg/kg), EEG/EMG parameters (wakefulness, NREM and REM sleep) were measured for 6 hours in four groups of rats. The first group were NP rats treated with CBD vehicle (n=8), the second group were NP rats treated with WAY 100635 alone at 2 mg/kg (n=6), the third group were NP rats treated with WAY 100635 (2 mg/kg)+CBD (20 mg/kg; n=6), and the fourth group were NP treated with CBD at 20 mg/kg (n=8). One-way ANOVA performed on the total time of wakefulness (F_{[3, 24]}=19.21, p<0.001) revealed a statistical difference between groups (FIG. 5C). Bonferroni post-hoc analysis did not detect any difference between NP rats treated with WAY 100635 (2 mg/kg)+CBD (20 mg/kg) and NP rats treated with vehicle, however, they display an increase in wakefulness relative to NP rats treated with CBD at 20 mg/kg (p<0.001). Moreover, the group treaded with WAY 100635 alone (2 mg/kg) show an increase in wakefulness relative to NP rats treated with vehicle (p<0.001) and NP rats treated with CBD at 20 mg/kg (p<0.001). One-way ANOVA between subjects performed on total NREM sleep revealed a significant main effect for groups (F_{[3, 24]}=18.85, p<0.001; FIG. 5B). Bonferroni post-hoc comparison analysis did not reveal any difference between NP rats treated with WAY 100635 (2 mg/kg) and CBD (20 mg/kg) and NP rats treated with vehicle or with NP rats treated with WAY 100635 alone (p>0.05). However, the WAY 100635+CBD group display a decrease in time spent in NREM sleep relative to NP rats treated with CBD at 20 mg/kg (P<0.001). Furthermore, WAY 100635 alone (2 mg/kg) statistically reduced the time spent NREM sleep relative to NP rats treated with vehicle (p>0.01). Additionally, One-way ANOVA between subjects performed on REM sleep revealed a statistical difference between groups (F_{[3, 24]}=10.27, p<0.001; FIG. 5A). Bonferroni post-hoc analysis showed that WAY 100635 (1 mg/kg) administration was able to block the positive effect of CBD (20 mg/kg) on REM sleep in NP rats (p<0.001), whereas no statistical difference was detected between NP rats treated with WAY 100635+CBD and NP treated with WAY 100635 alone. Furthermore, the post-hoc analysis showed that NP rats treated with WAY 100635 alone display a decrease in REM sleep when compared to NP rats treated with CBD at 20 mg/kg (p<0.01), whereas no statistical difference was detected between NP rats treated with vehicle and NP rats treated with WAY 100365 (2 mg/kg).

### Example 6: CBD Increases Power Spectra Intensity in NP Animals During NREM Sleep

To assess whether CBD (20 mg/kg) might affect the microarchitecture during the sleep, the intensity Delta (0.5-4 Hz), Theta (8-13 Hz) and Sigma (10-15 Hz) waves were measured during 6 hours of light phase in the 3 groups of rats (Naïve, SNI+VEH and SNI+CBD 20mg/kg). Data were converted as a percentage of variation vs. naive animals and One-way ANOVA was performed on the power of delta, theta and sigma waves during NREM and REN sleep.

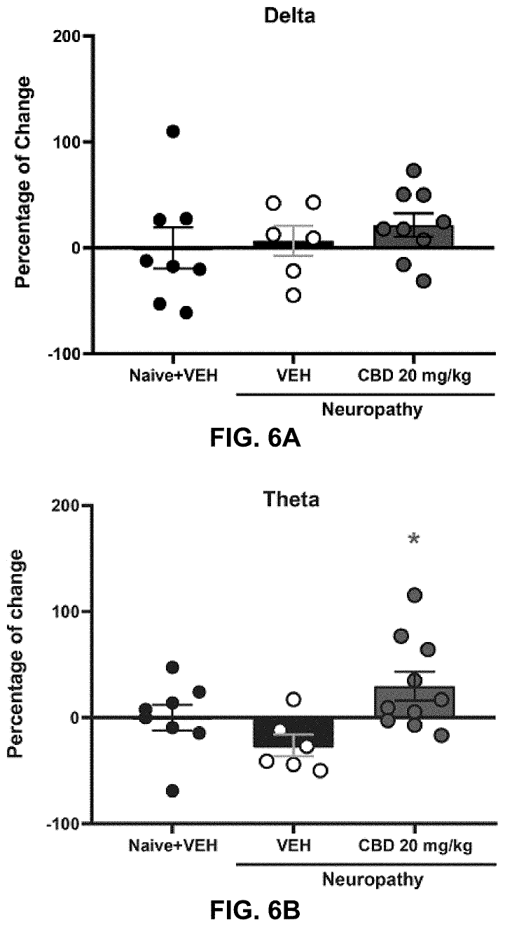

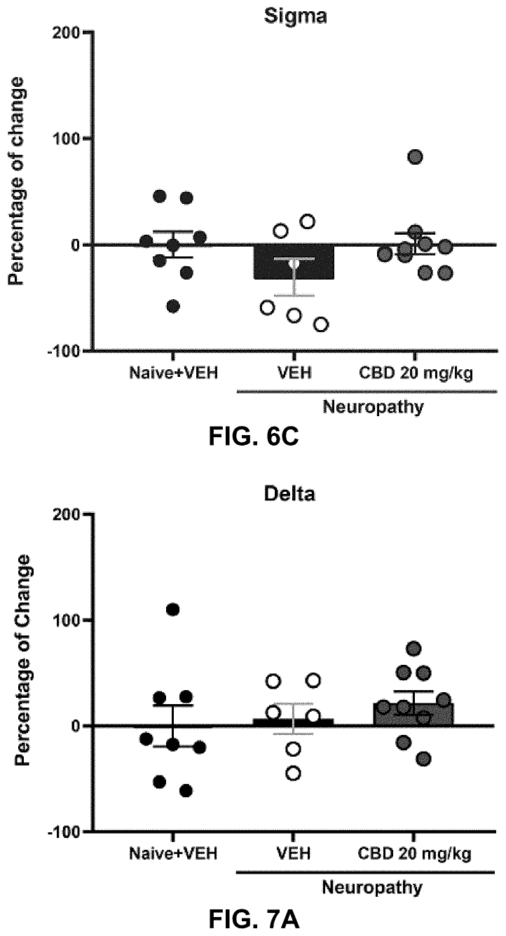

One-Way ANOVA between subjects on the power of Delta waves during the NREM sleep (F_{[2, 21]}=5.416, p<0.012) revealed a statistically significant difference between groups (FIG. 6A). Bonferroni post-hoc analysis shows that NP rats treated with CBD display an increase in the intensity of the power of Delta waves, relative to NP rats treated with vehicle (p<0.012), whereas no differences between naïve animals treated with vehicle and NP rats treated with vehicle were detected. One-way ANOVA of the groups on the power of theta waves during the NREM sleep (F_{[2, 21]}=4.549, p<0.022) revealed a significant difference between groups (FIG. 6B). Bonferroni post hoc reveal that CBD (20 mg/kg) administration in NP rats increases the intensity of the Theta waves relatively to NP animals treated with vehicle (p<0.022), no differences between naïve animals treated with vehicle and NP rats treated with vehicle were found. Additionally, one-way ANOVA between subjects performed on Sigma waves during NREM sleep revealed no significant main effects (F_{[2,21]}=1.766, p<0.1954, FIG. 6C).

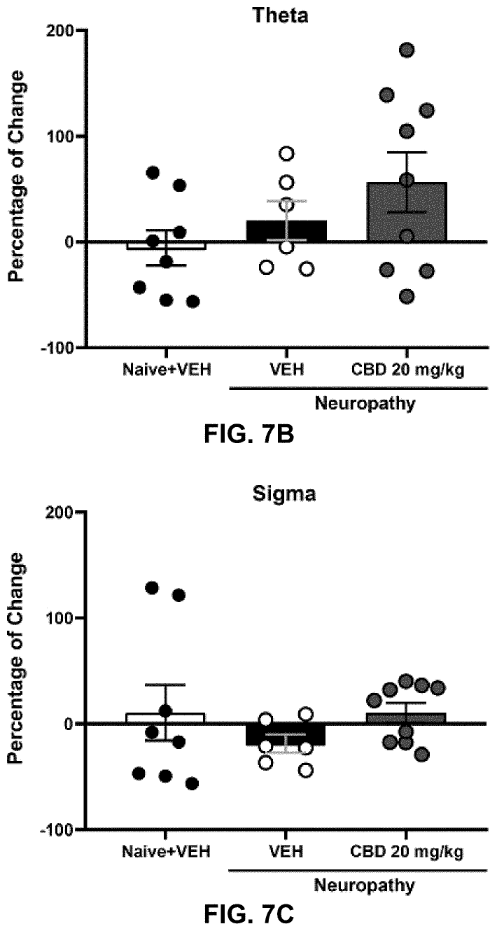

During REM sleep, One-Way ANOVA between subjects on the intensity of power of Delta (F_{[2, 20]}=5776, p<0.5703, FIG. 7A), Theta (F_{[2, 20]}=1.984, p<0.1636, FIG. 7B) and Sigma (F_{[2, 20]}=0.7902, p<0.4674) waves, revealed no statistically significant differences between groups (FIG. 7C).

To assess whether the 5-HT1A selective antagonist WAY 100635 (2 mg/kg) might reverse the effect of CBD (20 mg/kg) in the enhancement of power density (Delta, Theta, and Sigma waves) during sleep, the power spectra were measured for 6 hours in four groups of rats. The first group were NP rats treated with CBD vehicle (n=8), the second group were NP rats treated with WAY 100635 alone at 2 mg/kg (n=6), the third group were NP rats treated with WAY 100635 (2 mg/kg)+CBD (20 mg/kg; n=6), and the fourth group were NP treated with CBD at 20 mg/kg (n=8). Data were converted as a percentage of variation vs NP animals treated with vehicle.

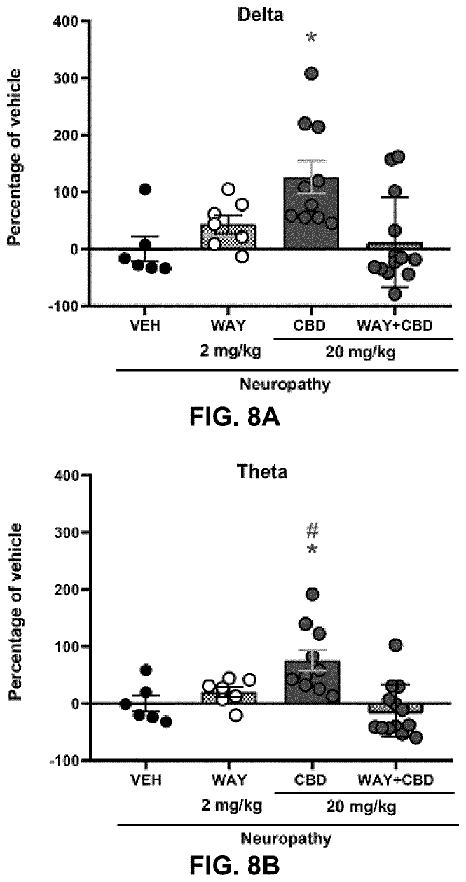

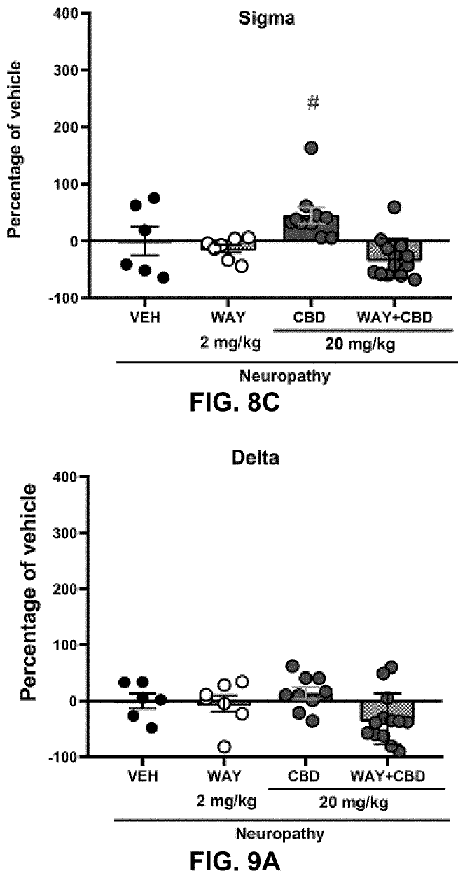

One-way ANOVA performed on the power intensity of Delta waves in NREM (F_{[3, 32]}=5.701, p<0.003) revealed a statistical difference between groups (FIG. 8A). Bonferroni post-hoc analysis showed that CBD (20 mg/kg) in NP rats induce an increase in the Delta power relatively to NP animals treated with vehicle (p<0.0132). Furthermore, WAY 100635 (2 mg/kg) administration was able to block the Delta power increase induced by CBD (20 mg/kg) on NREM sleep in NP rats (p<0.0048), while no statistical difference was detected between NP rats treated with WAY 100635+CBD and NP treated with WAY 100635 alone.

One-way ANOVA of the groups on the power of theta waves during the NREM sleep revealed a main effect (F_{[3, 32]}=7.752, p<0.0005, FIG. 8B). Bonferroni post hoc analysis showed that NP rats treated with CBD (20 mg/kg) display an increase in the theta power compared to NP animals treated with vehicle (p<0.0162). Additionally, WAY 100635 (2 mg/kg) administration was able to block the theta power increase induced by CBD (20 mg/kg) on NREM sleep in NP rats (p<0.0003). No statistical difference was detected between NP rats treated with WAY 100635+CBD and NP treated with WAY 100635 alone for theta.

Furthermore, one way ANOVA analysis on the intensity of Sigma waves (F_{[3, 32]}=6.831, p<0.0011) reveal a statistical difference between groups (FIG. 8C). Bonferroni post-hoc analysis did not detect differences between NP rats treated with CBD (20 mg/kg) and NP rats treated with vehicle, however, differences were detected between NP animals administrated with CBD (20 mg/kg) and NP rats treated with WAY 100635 (2 mg/kg) alone (p<0.038) and NP rats treated with WAY 100635+CBD (p<0.0006). Moreover, no statistical differences were detected between NP rats treated with WAY 100635+CBD and NP treated with Vehicle.

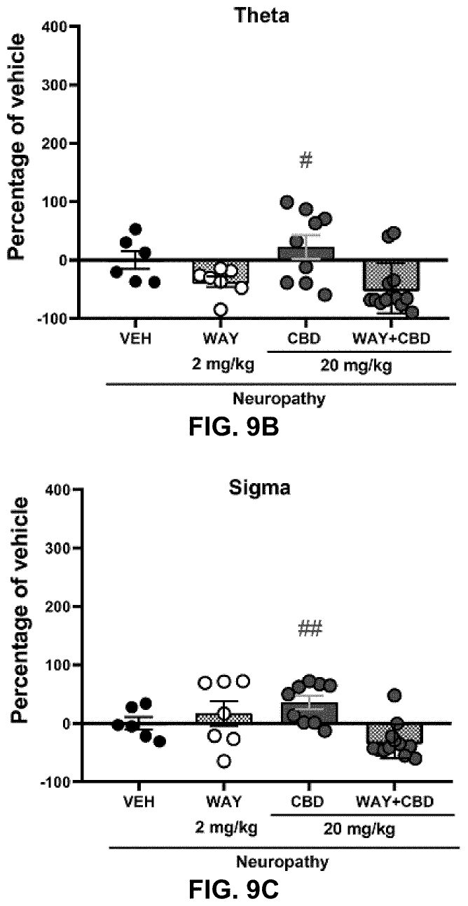

During REM sleep, one-Way ANOVA between subjects on the intensity of power of Delta (F_{[3, 31]}=2.672, p<0.0646), revealed no statistically significant differences between groups (FIG. 9A). One-way ANOVA analysis of the power of Theta waves during REM sleep reveal main statistical differences (F_{[3, 31]}=5.125, p<0.0054) between the groups (FIG. 9B). Bonferroni Post hoc shows no differences between the NP rats treated CBD (20 mg/kg) and NP animals treated with vehicle. However, ANOVA indicated that WAY 100635 (2 mg/kg) administration was able to block the positive effect of CBD (20 mg/kg) on theta waves during REM sleep in NP rats (p<0.0061). No statistical difference was detected between NP rats treated with WAY 100635+CBD and NP treated with WAY 100635 alone.

One-way ANOVA between subjects performed on the power of Sigma waves during REM sleep revealed a significant main effect for groups (F_{[3, 30]}=6.011, p<0.0025, FIG. 9 C). Bonferroni post-hoc comparison analysis reveals that WAY 100635 (2 mg/kg) blocks the effects of CBD (20 mg/kg) on Sigma power (p<0.0019). However, no differences were found between NP rats treated with CBD (20 mg) and NP rats treated with vehicle.

### Example 7: The CB1 Antagonist Rimonabant Does not Reverse the Sleep Perturbations Induced by Neuropathic Pain, but Decrease the Time Spent in REM

To evaluate whether Rimonabant (1 mg/kg) has an effect in the sleep of NP rats, EEG/EMG parameters (wakefulness, NREM and REM sleep) were measured for 6 hours in three groups of rats after i.p. rimonabant administration.

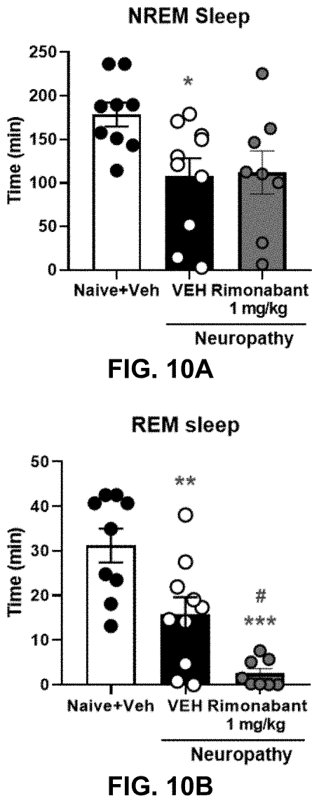

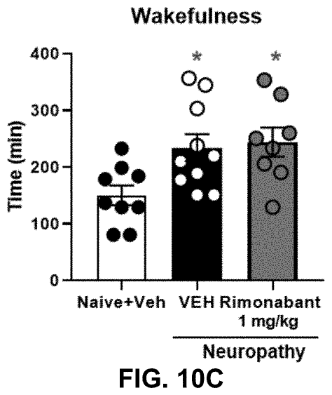

One-Way ANOVA for the 3 groups shows for NREM, (F_{[2, 24]}=4.023, p<0.05), Bonferroni's post-hoc test shows a difference of p<0.05 between Naïve+Veh vs. neuropathic+VEH. (FIG. 10A)

One-Way ANOVA for the 3 groups shows for REM, (F_{[2, 24]}=17.54, p<0.0001), Bonferroni's post-hoc test shows a difference of p<0.001 Naïve+Veh vs. neuropathic+VEH and p<0.0001. Naive+Veh vs. neuropathic+Rimonabant and p<0.05 Neuropathic+Veh vs. neuropathic+Rimonabant. (FIG. 10B)

One-Way ANOVA for the 3 groups shows for wakefulness, (F _{[2}, _{24]} =5.12, p <0.05) Bonferroni's post-hoc test shows a difference of p<0.05 Naïve+Veh vs. neuropathic+VEH and p<0.05 Naïve+Veh vs. neuropathic+Rimonabant (FIG. 10C).

This effect of the CB1 antagonist rimonabant on the reduction of REM sleep time provides evidence that CB1 antagonists may be useful for the treatment of disorders associated with dysregulated REM sleep such as narcolepsy and REM sleep behavior disorders (RBD).

Although the present invention has been described hereinabove by way of specific embodiments thereof, it can be modified, without departing from the spirit and nature of the subject invention as defined in the appended claims.

### REFERENCES

1. Colloca L, Ludman T, Bouhassira D, Baron R, Dickenson A H, Yarnitsky D, Freeman R, Truini A, Attal N, Finnerup N B. Neuropathic pain. Nature reviews Disease primers. 2017; 3(1):1-19.
2. Jensen T S, Finnerup N B. Allodynia and hyperalgesia in neuropathic pain: clinical manifestations and mechanisms. The Lancet Neurology. 2014; 13(9):924-935.
3. Chung K-F, Tso K-C. Relationship between insomnia and pain in major depressive disorder: A sleep diary and actigraphy study. Sleep medicine. 2010; 11(8):752-758.
4. Finan P H, Smith M T. The comorbidity of insomnia, chronic pain, and depression: dopamine as a putative mechanism. Sleep medicine reviews. 2013; 17(3):173-183.
5. Baron R. Mechanisms of disease: neuropathic pain—a clinical perspective. Nature clinical practice Neurology. 2006; 2(2):95-106.
6. von Hehn C A, Baron R, Woolf C J. Deconstructing the neuropathic pain phenotype to reveal neural mechanisms. Neuron. 2012; 73(4):638-652.
7. Cruccu G, Anand P, Attal N, Garcia-Larrea L, Haanpää M, JØrum E, Serra J, Jensen T. EFNS guidelines on neuropathic pain assessment. European journal of neurology. 2004; 11(3):153-162.
8. Tsuda M. New approach for investigating neuropathic allodynia by optogenetics. Pain. 2019; 160:553-558.
9. Petitjean H, Pawlowski S A, Fraine S L, Sharif B, Hamad D, Fatima T, Berg J, Brown C M, Jan L-Y, Ribeiro-da-Silva A. Dorsal horn parvalbumin neurons are gate-keepers of touch-evoked pain after nerve injury. Cell reports. 2015; 13(6):1246-1257.
10. Bair M J, Robinson R L, Katon W, Kroenke K. Depression and pain comorbidity: a literature review. Archives of internal medicine. 2003; 163(20):2433-2445.
11. Nicholson B, Verma S. Comorbidities in chronic neuropathic pain. Pain medicine. 2004; 5(suppl_1):59-527.
12. Fuller P M, Gooley J J, Saper C B. Neurobiology of the sleep-wake cycle: sleep architecture, circadian regulation, and regulatory feedback. Journal of biological rhythms. 2006;21(6):482-493.
13. Ochoa-Sanchez R, Comai S, Lacoste B, Bambico F R, Dominguez-Lopez S, Spadoni G, Rivara S, Bedini A, Angeloni D, Fraschini F. Promotion of non-rapid eye movement sleep and activation of reticular thalamic neurons by a novel MT2 melatonin receptor ligand. Journal of Neuroscience. 2011; 31(50): 18439-18452.
14. Finan P H, Goodin B R, Smith M T. The association of sleep and pain: an update and a path forward. The Journal of Pain. 2013; 14(12):1539-1552.
15. Gore M, Brandenburg N A, Dukes E, Hoffman D L, Tai K-S, Stacey B. Pain severity in diabetic peripheral neuropathy is associated with patient functioning, symptom levels of anxiety and depression, and sleep. Journal of pain and symptom management. 2005; 30(4):374-385.
16. Ferini-Strambi L. Neuropathic Pain and Sleep: A Review. Pain Ther. 2017; 6(Suppl 1):19-23.
17. Marshansky S, Mayer P, Rizzo D, Baltzan M, Denis R, Lavigne G J. Sleep, chronic pain, and opioid risk for apnea. Prog Neuropsychopharmacol Biol Psychiatry. 2018; 87(Pt B):234-244.
18. Monti J M. Hypnoticlike effects of cannabidiol in the rat. Psychopharmacology. 1977.
19. Chagas M H N, Crippa J A S, Zuardi A W, Hallak J E, Machado-de-Sousa J P, Hirotsu C, Maia L, Tufik S, Andersen M L. Effects of acute systemic administration of cannabidiol on sleep-wake cycle in rats. Journal of Psychopharmacology. 2013; 27(3):312-316.
20. Murillo-Rodriguez E, Millan-Aldaco D, Palomero-Rivero M, Mechoulam R, Drucker-Colín R. Cannabidiol, a constituent of Cannabis sativa, modulates sleep in rats. FEBS letters. 2006; 580(18):4337-4345.
21. Santucci V, Storme J-j, Soubrié P, Le Fur G. Arousal-enhancing properties of the CB1 cannabinoid receptor antagonist SR 141716A in rats as assessed by electroencephalographic spectral and sleep-waking cycle analysis. Life sciences. 1996; 58(6):PL103-PL110.
22. Perbal B. A practical guide to molecular cloning. 1988.
23. Sambrook J. Cold Spring. Molecular cloning: a laboratory manual. 1989.
24. Brown T A. Essential Molecular Biology: A Practical Approach Vol 1 2nd edition. 2000.
25. Glover D M. DNA cloning: a practical approach. Volume 1. 1985.
26. Ausubel F. Current Protocols in Molecular Biology. Greene Pub Associates: Wiley-Interscience New York; 1988.
27. Harlow E, Lane D. A laboratory manual. New York: Cold Spring Harbor Laboratory. 1988; 579.
28. Coligan J, Kruisbeek A, Margulies D, Shevach E, Strober W. Current Protocols in Immunology John Wiley & Sons. vol.-, No.-. 1998.
29. Mechoulam R, Hanuš L. Cannabidiol: an overview of some chemical and pharmacological aspects. Part I: chemical aspects. Chemistry and physics of lipids. 2002; 121(1-2):35-43.
30. Jones P G, Falvello L, Kennard O, Sheldrick G, Mechoulam R. Cannabidiol. Acta Crystallographica Section B: Structural Crystallography and Crystal Chemistry. 1977; 33(10):3211-3214.
31. ElSohly M A, Slade D. Chemical constituents of marijuana: the complex mixture of natural cannabinoids. Life sciences. 2005; 78(5):539-548.
32. American Psychiatric Association A. Diagnostic and statistical manual of mental disorders. Vol 5: American Psychiatric Association Washington, D.C.; 1980.
33. Remington J P. Remington: the science and practice of pharmacy. Vol 1: Lippincott Williams & Wilkins; 2006.
34. Rowe R, Sheskey P, Cook W, Fenton M. Association AP. Handbook of pharmaceutical excipients: London: Pharmaceutical Press; 2012.
35. Decosterd I, Woolf C J. Spared nerve injury: an animal model of persistent peripheral neuropathic pain. Pain. 2000; 87(2):149-158.
36. De Gregorio D, McLaughlin R J, Posa L, Ochoa-Sanchez R, Enns J, Lopez-Canul M, Aboud M, Maione S, Comai S, Gobbi G. Cannabidiol modulates serotonergic transmission and reverses both allodynia and anxiety-like behavior in a model of neuropathic pain. Pain. 2019; 160(1):136.
37. Lopez-Canul M, Palazzo E, Dominguez-Lopez S, Luongo L, Lacoste B, Comai S, Angeloni D, Fraschini F, Boccella S, Spadoni G. Selective melatonin MT2 receptor ligands relieve neuropathic pain through modulation of brainstem descending antinociceptive pathways. Pain. 2015; 156(2):305-317.
38. Chaplan S R, Bach F, Pogrel J, Chung J, Yaksh T. Quantitative assessment of tactile allodynia in the rat paw. Journal of neuroscience methods. 1994; 53(1):55-63.
39. Dixon W J. Efficient analysis of experimental observations. Annual review of pharmacology and toxicology. 1980; 20(1):441-462.
40. Haba-Rubio J, Ibanez V, Sforza E. An alternative measure of sleep fragmentation in clinical practice: the sleep fragmentation index. Sleep medicine. 2004; 5(6):577-581.
41. Jo Nijs, Olivier Mairesse, Daniel Neu, Laurence Leysen, Lieven Danneels, Barbara Cagnie, Mira Meeus, Maarten Moens, Kelly Ickmans, Dorien Goubert. Physical Therapy, Volume 98, Issue 5, May 2018, Pages 325-335.

What is claimed is:

1. A method for treating or managing insomnia or a sleep disorder associated to pain in a human subject in need thereof, the method comprising administering to the subject an effective amount of cannabidiol (CBD), a CBD analog, or a pharmaceutically acceptable salt or solvate thereof.

2. The method of claim 1, wherein the pain is neuropathic pain.

3. The method of claim 2, wherein the neuropathic pain is post-herpetic (or post-shingles) neuralgia, reflex sympathetic dystrophy/causalgia (nerve trauma), components of cancer pain, phantom limb pain, entrapment neuropathy, peripheral neuropathy (widespread nerve damage), diabetic neuropathy, lower back pain, pain induced by chemotherapy (cisplatin, paclitaxel, vincristine, etc.) or radiotherapy, pain caused by HIV infection or AIDS, pain caused by central nervous system disorders, complex regional pain syndrome, nerve compression or infiltration by tumors.

4. The method of claim 1, wherein the CBD analog is cannabidiolic acid (CBDA), cannabidiol-3-monomethyl ether (CBDM-C₅), cannabidibutol (CBD-C₄), cannabidivarinic acid (CBDVA), cannabidivarin (CBDV), or cannabidiorcol (CBD-C₁).

5. The method of claim 1, wherein the method comprises administering to the subject an effective amount of CBD or a pharmaceutically acceptable salt or solvate thereof.

6. The method of claim 5, wherein the method comprises administering to the subject an effective amount of CBD.

7. The method of claim 1, wherein the CBD, CBD analog, or pharmaceutically acceptable salt or solvate thereof is administered to the human subject at a dose corresponding to a dose of about 5 to about 20 milligrams/kg in rats.

8. The method of claim 1, wherein the total daily dose of the CBD, CBD analog, or pharmaceutically acceptable salt or solvate thereof administered to the human subject corresponding to a dose of about 5 to about 20 milligrams/kg in rats.

9. The method of claim 1, wherein the CBD, CBD analog, or pharmaceutically acceptable salt or solvate thereof is administered once-a-day or twice-a-day.

10. The method of claim 1, wherein the CBD, CBD analog, or pharmaceutically acceptable salt or solvate thereof is administered for at least a week.

11. The method of claim 1, wherein the CBD, CBD analog, or pharmaceutically acceptable salt or solvate thereof is administered as an immediate release formulation, a controlled release formulation, and/or an extended release formulation.

12. The method of claim 11, wherein the CBD, CBD analog, or pharmaceutically acceptable salt or solvate thereof is administered parentally or orally.

13. The method of claim 1, wherein the method (a) increases REM sleep time, (b) increases non-REM (NREM) sleep and/or SWS time, and/or (c) decrease wakefulness time in the subject.

14. A method for identifying a subject suffering from insomnia or a sleep disorder associated to pain, the method comprising:
measuring at least one of the following parameters in the subject: rapid eye movement (REM) sleep time, non-REM sleep or Slow Wave Sleep (SWS) time, and wakefulness time; and
comparing the measured REM sleep time, non-REM sleep or SWS time, and/or
wakefulness time to corresponding control or reference times;
wherein a reduced (shorter) REM sleep time, reduced (shorter) non-REM sleep and/or SWS time and/or increased (longer) wakefulness time relative to the corresponding control or reference times is indicative that the subject suffers from insomnia or a sleep disorder associated to pain.

15. A method for treating a disorder associated with abnormal rapid eye movement (REM) sleep in a subject, the method comprising administering to the subject an effective amount of a cannabinoid receptor 1 (CB₁) inhibitor.

16. The method of claim 15, wherein the abnormal REM sleep is excessive REM sleep.

17. The method of claim 15, wherein the disorder is narcolepsy or a REM sleep behavior disorder (RBD).

18. The method of claim 17, wherein the RBD is associated with Lewy body dementia, Parkinson's disease or multiple system atrophy.

19. The method of claim 15, wherein the CB₁ inhibitor is a CB₁ inverse agonist.

20. The use of claim 15, wherein the CB₁ inhibitor is Rimonabant or an analog thereof.
